(19) United States
(12) Patent Application Publication
(10) Pub. No.: US 2010/0056520 A1
(43) Pub. Date: Mar. 4, 2010

(54) FORMULATIONS OF PHOSPHOLIPASE ENZYME INHIBITORS

(76) Inventors: Frances Anne Donahue, Garfield, NJ (US); Marc Sadler Tesconi, Monroe, NY (US); Mannching Sherry Ku, Thiells, NY (US)

Correspondence Address: WYETH LLC;PATENT LAW GROUP, 5 GIRALDA FARMS, MADISON, NJ, 07940, US

(21) Appl. No.: 12/513,008

(22) PCT Filed: Oct. 30, 2007
(86) PCT No.: PCT/US07/82982

§ 371 Date: Apr. 30, 2009

Related U.S. Application Data
(60) Provisional application No. 60/855,569, filed on Oct. 31, 2006.

(51) Int. Cl. A61K 31/5377; A61K 31/4025; A61K 31/497; A61P 25/00

(52) U.S. Cl. 514/235.2; 514/419; 514/254.08

(57) ABSTRACT

The present invention is directed to formulations of inhibitors of phospholipase enzymes, such as cytosolic PLA₂, compositions containing the same and processes for manufacture thereof.

### CROSS REFERENCE TO RELATED APPLICATIONS

This application claims the benefit of U.S. Provisional Application No. 60/855,569, filed on Oct. 31, 2006, which is incorporated herein by reference in its entirety.

### FIELD OF THE INVENTION

The present invention is directed to formulations of inhibitors of phospholipase enzymes, such as cytosolic PLA₂, compositions containing the same and processes for manufacture thereof.

### BACKGROUND OF THE INVENTION

Leukotrienes and prostaglandins are important mediators of inflammation, each of which contributes to the development of an inflammatory response in a different way. Leukotrienes recruit inflammatory cells such as neutrophils to an inflamed site, promote the extravasation of these cells and stimulate release of superoxide and proteases, which damage the tissue. Leukotrienes also play a pathophysiological role in the hypersensitivity experienced by asthmatics [See, e.g. B. Samuelson et al., Science, 237:1171-76 (1987)]. Prostaglandins enhance inflammation by increasing blood flow and therefore infiltration of leukocytes to inflamed sites. Prostaglandins also potentiate the pain response induced by stimuli.

Prostaglandins and leukotrienes are unstable and are not stored in cells, but are instead synthesized [W. L. Smith, Biochem. J., 259:315-324 (1989)] from arachidonic acid in response to stimuli. Prostaglandins are produced from arachidonic acid by the action of COX-1 and COX-2 enzymes. Arachidonic acid is also the substrate for the distinct enzyme pathway leading to the production of leukotrienes.

Arachidonic acid, which is fed into these two distinct inflammatory pathways, is released from the sn-2 position of membrane phospholipids by phospholipase A₂ enzymes (hereinafter PLA₂). The reaction catalyzed by PLA₂ is believed to represent the rate-limiting step in the process of lipid mediated biosynthesis and the production of inflammatory prostaglandins and leukotrienes. When the phospholipid substrate of PLA₂ is of the phosphotidyl choline class with an ether linkage in the sn-1 position, the lysophospholipid produced is the immediate precursor of platelet activating factor (hereafter called PAF), another potent mediator of inflammation [S. I. Wasserman, Hospital Practice, 15:49-58 (1988)].

Most anti-inflammatory therapies have focused on preventing production of either prostaglandins or leukotrienes from these distinct pathways, but not on all of them. For example, ibuprofen, aspirin, and indomethacin are all NSAIDs, which inhibit the production of prostaglandins by COX-1/COX-2 inhibition, but have no effect on the inflammatory production of leukotrienes from arachidonic acid in the other pathways. Conversely, zileuton inhibits only the pathway of conversion of arachidonic acid to leukotrienes, without affecting the production of prostaglandins. None of these widely-used anti-inflammatory agents affects the production of PAF.

Consequently the direct inhibition of the activity of PLA₂ has been suggested as a useful mechanism for a therapeutic agent, i.e., to interfere with the inflammatory response. [See, e.g., J. Chang et al, Biochem. Pharmacol., 36:2429-2436 (1987)].

A family of PLA₂ enzymes characterized by the presence of a secretion signal sequenced and ultimately secreted from the cell have been sequenced and structurally defined. These secreted PLA₂s have an approximately 14 kD molecular weight and contain seven disulfide bonds, which are necessary for activity. These PLA₂s are found in large quantities in mammalian pancreas, bee venom, and various snake venom. [See, e.g., references 13-15 in Chang et al, cited above; and E. A. Dennis, Drug Devel. Res., 10:205-220 (1987).] However, the pancreatic enzyme is believed to serve a digestive function and, as such, should not be important in the production of the inflammatory mediators whose production must be tightly regulated.

The primary structure of the first human non-pancreatic PLA₂ has been determined. This non-pancreatic PLA₂ is found in platelets, synovial fluid, and spleen and is also a secreted enzyme. This enzyme is a member of the aforementioned family. [See, J. J. Seilhamer et al, J. Biol. Chem., 264:5335-5338 (1989); R. M. Kramer et al, J. Biol. Chem., 264:5768-5775 (1989); and A. Kando et al, Biochem. Biophys. Res. Comm., 163:42-48 (1989)]. However, it is doubtful that this enzyme is important in the synthesis of prostaglandins, leukotrienes and PAF, since the non-pancreatic PLA₂ is an extracellular protein, which would be difficult to regulate, and the next enzymes in the biosynthetic pathways for these compounds are intracellular proteins. Moreover, there is evidence that PLA₂ is regulated by protein kinase C and G proteins [R. Burch and J. Axelrod, Proc. Natl. Acad. Sci. U.S.A., 84:6374-6378 (1989)], which are cytosolic proteins, which must act on intracellular proteins. It would be impossible for the non-pancreatic PLA₂ to function in the cytosol, since the high reduction potential would reduce the disulfide bonds and inactivate the enzyme.

A murine PLA₂ has been identified in the murine macrophage cell line, designated RAW 264.7. A specific activity of 2 mols/min/mg, resistant to reducing conditions, was reported to be associated with the approximately 60 kD molecule. However, this protein was not purified to homogeneity. [See, C. C. Leslie et al, Biochem. Biophys. Acta., 963:476-492 (1988)]. The references cited above are incorporated by reference herein for information pertaining to the function of the phospholipase enzymes, particularly PLA₂.

A cytosolic phospholipase A₂ alpha (hereinafter “cPLA₂α”) has also been identified and cloned. See, U.S. Pat. Nos. 5,322,776 and 5,354,677, which are incorporated herein by reference as if fully set forth. The enzyme of these patents is an intracellular PLA₂ enzyme, purified from its natural source or otherwise produced in purified form, which functions intracellularly to produce arachidonic acid in response to inflammatory stimuli.

In addition to the identification of several phospholipase enzymes, efforts have been spent in identifying chemical inhibitors of the action of specific phospholipase enzymes, which inhibitors could be used to treat inflammatory conditions, particularly where inhibition of production of prostaglandins, leukotrienes and PAF are all desired results. Such inhibitors are disclosed, for example, in U.S. Pat. No. 6,797,708 and U.S. patent application Ser. No. 11/442,199 (filed May 26, 2006), each of which is incorporated herein by reference in their entireties.

Given the importance of these compounds as pharmaceutical agents, it can be seen that effective formulations for delivery of the compounds, including those having improved bioavailability, are of great import, and there is an ongoing need for such new formulations.

### SUMMARY OF THE INVENTION

The present invention provides pharmaceutical compositions comprising:
a) a pharmaceutically effective amount of an active pharmacological agent having the Formula I:

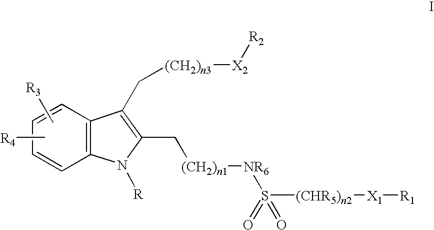

or a pharmaceutically acceptable salt thereof, wherein R, R₁, R₂, R₃, R₄, R₆, X₁, X₂, n₁, n₂, and n₃ are defined as described herein; and
b) a carrier or excipient system comprising a first solubilizer, a second solubilizer, and a diluent.

The present invention also provides pharmaceutical compositions comprising:

a) a pharmaceutically effective amount of an active pharmacological agent having the Formula II:

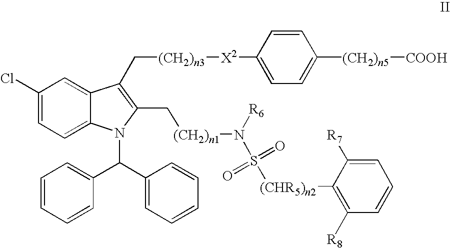

and pharmaceutically acceptable salts thereof, wherein R₅, R₆, R₇, R₈, X², n₁, n₂, n₃, and n₅ are defined as described herein; and

b) a carrier or excipient system comprising a first solubilizer, a second solubilizer, and a diluent.

The invention further provides processes for preparing the pharmaceutical compositions and dosage forms of the invention, and products of the processes.

### BRIEF DESCRIPTION OF THE DRAWINGS

FIG. 1 is a graph depicting the dissolution profile of a formulation according to the invention at different pH.

FIG. 2 is a graph depicting the dissolution profiles of a formulation according to the invention (□) and the corresponding encapsulated active pharmacological agent having Formula I (----·----).

FIG. 3 is a graph depicting a comparison of AUC (0−t)/Dose of a formulation according to the invention in fed versus fasted dogs.

FIG. 4 is a graph depicting the dissolution profiles of a formulation according to the invention (▪ and Δ) and the corresponding encapsulated active pharmacological agent having Formula I (♦).

### DETAILED DESCRIPTION OF THE INVENTION

In one aspect of the invention, a pharmaceutical composition comprises
a) a pharmaceutically effective amount of an active pharmacological agent having Formula I:

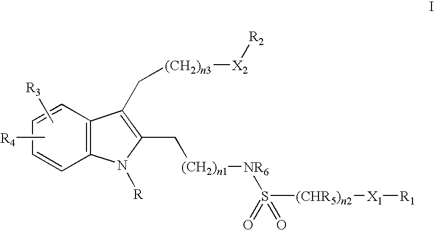

or a pharmaceutically acceptable salt thereof, wherein:
R is selected from the formulae —(CH₂)ₙ-A, —(CH₂)ₙ—S-A, and —(CH₂)ₙ—O-A, wherein A is selected from the moieties:

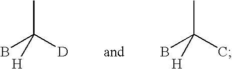

wherein

D is C₁-C₆ alkyl, C₁-C₆ alkoxy, C₃-C₆ cylcoalkyl, —CF₃, or —(CH₂)₁₋₃—CF₃;

B and C are independently selected from phenyl, pyridinyl, pyrimidinyl, furyl, thienyl and pyrrolyl groups, each optionally substituted by from 1 to 3, preferably 1 to 2, substituents selected independently from halogen, —CN, —CHO, —CF₃, —OCF₃, —OH, C₁-C₆ alkyl, C₁-C₆ alkoxy, —NH₂, —N(C₁-C₆ alkyl)₂, —NH(C₁-C₆ alkyl), —NH—C(O)—(C₁-C₆ alkyl), —NO₂, or by a 5- or 6-membered heterocyclic or heteroaromatic ring containing 1 or 2 heteroatoms selected from O, N, and S; or
n is an integer from 0 to 3;
n₁ is an integer from 1 to 3;
n₂ is an integer from 0 to 4;
n₃ is an integer from 0 to 3;
n₄ is an integer from 0 to 2;
X₁ is selected from a chemical bond, —S—, —O—, —S(O)—, —S(O)₂—, —NH—, —C═C—,

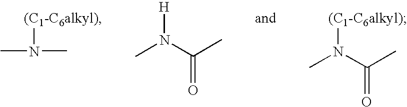

R₁ is selected from C₁-C₆ alkyl, C₁-C₆ fluorinated alkyl, C₃-C₆ cycloalkyl, tetrahydropyranyl, camphoryl, adamantyl, —CN, —N(C₁-C₆ alkyl)₂, phenyl, pyridinyl, pyrimidinyl, furyl, thienyl, napthyl, morpholinyl, triazolyl, pyrazolyl, piperidinyl, pyrrolidinyl, imidazolyl, piperizinyl, thiazolidinyl, thiomorpholinyl, tetrazolyl, indolyl, benzoxazolyl, benzofuranyl, imidazolidine-2-thionyl, 7,7-dimethyl-bicyclo[2.2.1]heptan-2-onyl, benzo[1,2,5]oxadiazolyl, 2-oxa-5-aza-bicyclo[2.2.1]heptanyl, piperazin-2-onyl and pyrrolyl groups, each optionally substituted by from 1 to 3, preferably 1 to 2, substituents independently selected from halogen, —CN, —CHO, —CF₃, —OCF₃, —OH, C₁-C₆ alkyl, C₁-C₆ alkoxy, —NH₂, —N(C₁-C₆ alkyl)₂, —NH(C₁-C₆ alkyl), —NH—C(O)—(C₁-C₆ alkyl), —NO₂, —SO₂(C₁-C₃ alkyl), —SO₂NH₂, —SO₂NH(C₁-C₃ alkyl), —SO₂N(C₁-C₃ alkyl)₂, —COOH, —CH₂—COOH, —CH₂—NH(C₁-C₆ alkyl), —CH₂—N(C₁-C₆ alkyl)₂, —CH₂—NH₂, pyridinyl, 2-methyl-thiazolyl, morpholino, 1-chloro-2-methyl-propyl, C₁-C₆ thioalkyl, phenyl (further optionally substituted with one or more (e.g., 1-5, 1-4, 1-3, or 1-2) halogens), dialkylamino, —CN or —OCF₃), benzyloxy, —(C₁-C₃ alkyl)C(O)CH₃, —(C₁-C₃ alkyl)OCH₃, —C(O)NH₂, or

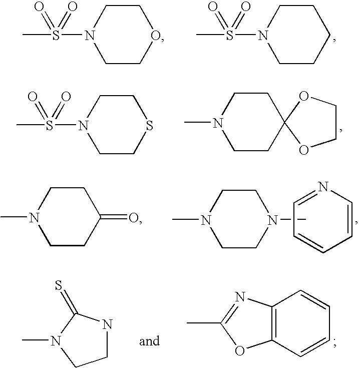

X₂ is selected from —O—, —CH₂—, —S—, —SO—, —SO₂—, —NH—, —C(O)—,

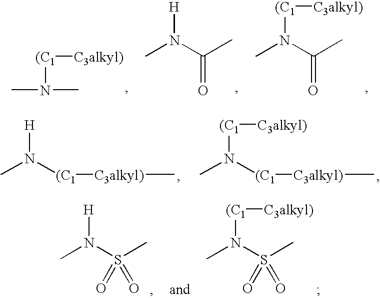

R₂ is a ring moiety selected from phenyl, pyridinyl, pyrimidinyl, furyl, thienyl and pyrrolyl groups, the ring moiety being substituted by a group of the formula —(CH₂)ₙ₄—CO₂H or a pharmaceutically acceptable acid mimic or mimetic; and also optionally substituted by 1 or 2 additional substituents independently selected from halogen, —CN, —CHO, —CF₃, —OCF₃, —OH, C₁-C₆ alkyl, C₁-C₆ alkoxy, C₁-C₆ thioalkyl, —NH₂, —N(C₁-C₆ alkyl)₂, —NH(C₁-C₆ alkyl), —NH—C(O)—(C₁-C₆ alkyl), and —NO₂;

R₃ is selected from H, halogen, —CN, —CHO, —CF₃, —OCF₃, —OH, C₁-C₆ alkyl, C₁-C₆ alkoxy, C₁-C₆ thioalkyl, —NH₂, —N(C₁-C₆ alkyl)₂, —NH(C₁-C₆ alkyl), —NH—C(O)—(C₁-C₆ alkyl), and —NO₂;

R₄ is selected from H, halogen, —CN, —CHO, —CF₃, —OCF₃, —OH, C₁-C₆ alkyl, C₁-C₆ alkoxy, C₁-C₆ thioalkyl, —NH₂, —N(C₁-C₆ alkyl)₂, —NH(C₁-C₆ alkyl), —NH—C(O)—(C₁-C₆ alkyl), —NO₂, —NH—C(O)—N(C₁-C₃ alkyl)₂, —NH—C(O)—NH(C₁-C₃ alkyl), —NH—C(O)—O—(C₁-C₃ alkyl), —SO₂—C₁-C₆ alkyl, —S—C₃-C₆ cycloalkyl, —S—CH₂—C₃-C₆ cycloalkyl, —SO₂—C₃-C₆ cycloalkyl, —SO₂—CH₂—C₃-C₆ cycloalkyl, C₃-C₆ cycloalkyl, —CH₂—C₃-C₆ cycloalkyl, —O—C₃-C₆ cycloalkyl, —O—CH₂—C₃-C₆ cycloalkyl, phenyl, benzyl, benzyloxy, morpholino, pyrrolidino, piperidinyl, piperizinyl, furanyl, thienyl, imidazolyl, tetrazolyl, pyrazinyl, pyrazolonyl, pyrazolyl, oxazolyl, and isoxazolyl, the rings of each of these R₄ groups each being optionally substituted by from 1 to 3 substituents selected from the group of halogen, —CN, —CHO, —CF₃, —OH, C₁-C₆ alkyl, C₁-C₆ alkoxy, —NH₂, —N(C₁-C₆ alkyl)₂, —NH(C₁-C₆ alkyl), —NH—C(O)—(C₁-C₆ alkyl), —NO₂, —SO₂(C₁-C₃ alkyl), —SO₂NH(C₁-C₃ alkyl), —SO₂N(C₁-C₃ alkyl)₂, and —OCF₃;

each R₅ is independently H or C₁₋₃ alkyl; and

R₆ is H or C₁₋₆ alkyl; and
b) a carrier or excipient system comprising:
i) about 10 to about 50% a first solubilizer by weight of the composition;
ii) about 10 to about 50% a second solubilizer by weight of the composition; and
iii) about 10 to about 30% a diluent by weight of the composition.

In some embodiments, the pharmaceutical composition described above comprises the pharmacologically active agent wherein

R₁ is optionally substituted phenyl; and

R is

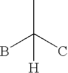

where B and C are phenyl.

In one aspect, this invention provides pharmaceutical compositions comprising:

a) a pharmaceutically effective amount of an active pharmacological agent having Formula II:

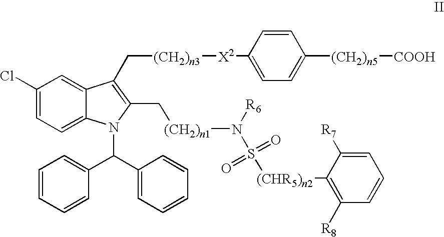

or a pharmaceutically acceptable salt thereof, wherein:

n₁ is 1 or 2;

n₂ is 1 or 2;

n₃ is 1 or 2;

n₅ is 0, 1 or 2;

X² is O, —CH₂— or SO₂;

each R₅ is independently H or C₁₋₃ alkyl;

R₆ is H or C₁₋₆ alkyl;

R₇ is selected from the group consisting of —OH, benzyloxy, —CH₃, —CF₃, —OCF₃, C₁₋₃ alkoxy, halogen, —CHO, —CO(C₁₋₃ alkyl), —CO(OC₁₋₃ alkyl), quinoline-5-yl, 3,5-dimethylisoxazol-4-yl, thiophene-3-yl, pyridin-4-yl, pyridine-3-yl, —CH₂-Q, and phenyl optionally substituted by from one to three independently selected R₃₀ groups;

R₈ is selected from the group consisting of H, —OH, —NO₂, —CF₃, —OCF₃, C₁₋₃ alkoxy, halogen, —CO(C₁₋₃ alkyl), —CO(OC₁₋₃ alkyl), quinoline-5-yl, 3,5-dimethylisoxazol-4-yl, thiophene-3-yl, —CH₂-Q, and phenyl substituted by from one to three independently selected R₃₀ groups;

Q is OH, dialkylamino,

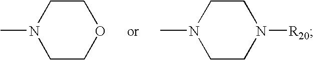

R₂₀ is selected from the group consisting of H, C₁₋₃ alkyl, and —CO(C₁₋₃ alkyl); and

R₃₀ is selected from the group consisting of dialkylamino, —CN, and —OCF₃;

provided that:

i) when each R₅ is H, R₆ is H, n₅ is 0, and R₈ is H, then R₇ cannot be chlorine;

ii) when each R₅ is H, R₆ is H, n₅ is 0, X² is O or —CH₂—, and R₈ is H, then R₇ cannot be CH₃;

iii) when each R₅ is H, and R₆ is H, then R₇ and R₈ cannot both be fluorine;

iv) when each R₅ is H, R₆ is H, and X² is O, then R₇ and R₈ cannot both be chlorine;

v) when each R₅ is H, R₆ is H, X² is O, and R₈ is NO₂, then R₇ cannot be fluorine; and

vi) when each R₅ is H, R₆ is H, X² is SO₂, and R₈ is H, then R₇ cannot be fluorine or chlorine; and

b) a carrier or excipient system comprising:
i) about 10 to about 50% a first solubilizer by weight of the composition;
ii) about 10 to about 50% a second solubilizer by weight of the composition; and
iii) about 10 to about 30% a diluent by weight of the composition.

In some embodiments, the compound of Formula II has the Formula III:

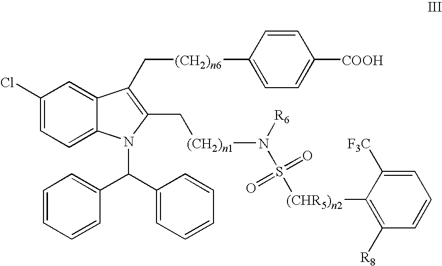

or a pharmaceutically acceptable salt thereof, wherein:

n₁ is 1 or 2;

n₂ is 1 or 2;

n₆ is 1 or 2;

R₅ is H or CH₃;

R₆ is H or C₁₋₆ alkyl; and

R₈ is selected from the group consisting of H, —OH, —NO₂, —CF₃, —OCF₃, —OCH₃, halogen, —COCH₃, —COOCH₃, dimethylamino, diethylamino, and —CN.

In some further embodiments, the compound of Formula I or Formula II is 4-(3-{5-chloro-1-(diphenylmethyl)-2-[2-({[2-(trifluoromethyl)benzyl]sulfonyl}amino)ethyl]-1H-indol-3-yl}propyl)benzoic acid or a pharmaceutically acceptable salt thereof.

It will be understood that the C₁-C₆ fluorinated alkyl groups in the definition of R₁ may be any alkyl group of 1 to 6 carbon atoms with any amount of fluorine substitution including, but not limited to, —CF₃, alkyl chains of 1 to 6 carbon atoms terminating in a trifluoromethyl group, —CF₂CF₃, etc.

As used herein, the terms “heterocyclic” and “heterocyclyl” refer to a saturated or partially unsaturated (nonaromatic) monocyclic, bicyclic, tricyclic or other polycyclic ring system having 1-4 ring heteroatoms if monocyclic, 1-8 ring heteroatoms if bicyclic, or 1-10 ring heteroatoms if tricyclic, each of said heteroatoms being independently selected from O, N, and S (and mono and dioxides thereof, e.g., NO→O—, S(O), SO₂. A ring heteroatom or a ring carbon can serve as the point of attachment of the heterocyclic ring to another moiety. Any atom can be substituted, e.g., by one or more substituents. Heterocyclyl groups can include, e.g. and without limitation, tetrahydropyranyl, piperidyl (piperidino), piperazinyl, morpholinyl (morpholino), thiomorpholinyl, pyrrolinyl, and pyrrolidinyl.

The term “heteroaromatic” refers to an aromatic monocyclic, bicyclic, tricyclic, or other polycyclic hydrocarbon group having 1-4 ring heteroatoms if monocyclic, 1-8 ring heteroatoms if bicyclic, or 1-10 ring heteroatoms if tricyclic, each of said heteroatoms being independently selected from O, N, and S (and mono and dioxides thereof, e.g., N→O⁻, S(O), SO₂). Any atom can be substituted, e.g., by one or more substituents. Heteroaromatic rings can include, e.g. and without limitation, pyridinyl, thiophenyl (thienyl), furyl (furanyl), imidazolyl, indolyl, isoquinolyl, quinolyl and pyrrolyl.

Pharmaceutically acceptable acid mimics or mimetics useful in the compounds of this invention include those wherein R₂ is selected from the group of:

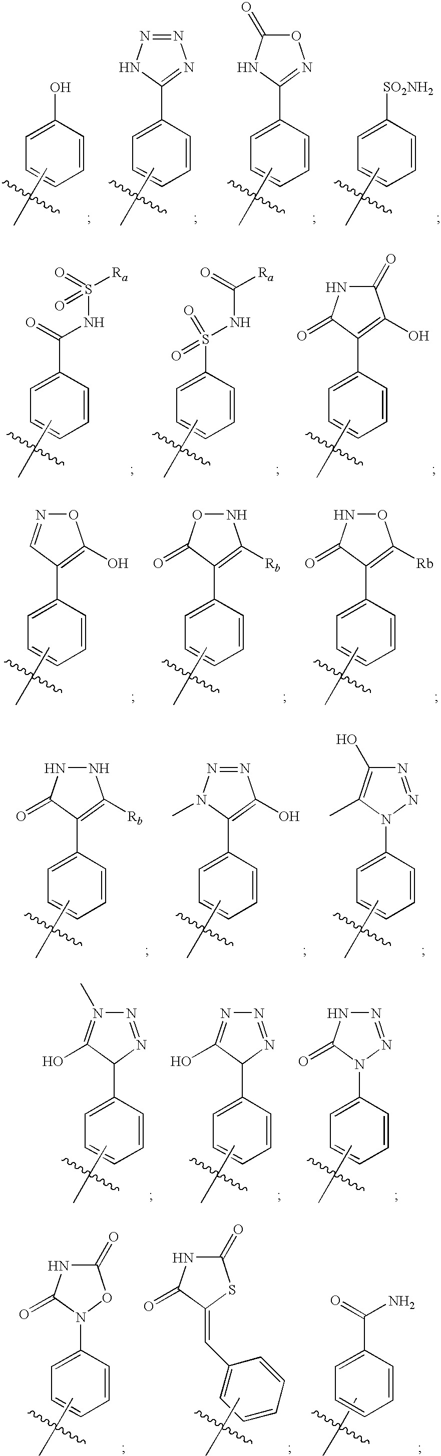

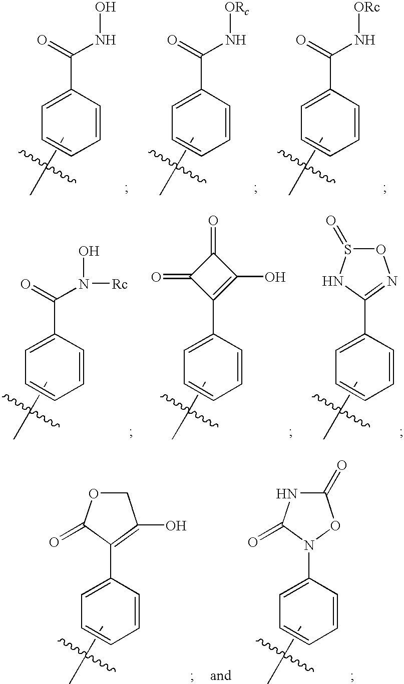

wherein Rₐ is selected from —CF₃, —CH₃, phenyl, and benzyl, with the phenyl or benzyl groups being optionally substituted by from 1 to 3 groups selected from C₁-C₆ alkyl, C₁-C₆ alkoxy, C₁-C₆ thioalkyl, —CF₃, halogen, —OH, and —COOH; R_{b} is selected from —CF₃, —CH₃, —NH₂, phenyl, and benzyl, with the phenyl or benzyl groups being optionally substituted by from 1 to 3 groups selected from C₁-C₆ alkyl, C₁-C₆ alkoxy, C₁-C₆ thioalkyl, —CF₃, halogen, —OH, and —COOH; and R_{c} is selected from —CF₃ and C₁-C₆ alkyl.

Those of skill in the art will be able to readily ascertain pharmaceutically effective amounts of said active pharmacological agent. Generally, the active pharmacological agent is present in the composition in an amount of from about 0.1% to about 25% by weight of the composition.

In some embodiments, the invention provides unit dosage forms containing the compositions of the invention. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Thus, the unit dosage forms formulations of the present invention include any conventionally used forms, including capsules, gels, oral liquids, and the like. In some embodiments, the unit dosage form is a capsule.

As will be recognized, a unit dosage form, such as a capsule, tablet, or other dosage form, will generally contain a pharmaceutically effective amount of the active pharmacological agent. As will be recognized, the pharmacological agent can be effective over a wide dosage range, and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

Generally, on a weight basis, the pharmaceutically effective amount is from about 1 mg to about 125 mg of active pharmacological agent. Thus, the unit dosage forms of the invention can contain various doses of the active pharmacological agent, for example approximate doses of 5, 10, 25, 50, 75, 100 and 125 mg, as well as others. Accordingly, the invention includes dosage forms that contain pharmaceutical compositions of the invention, that include from about 3 mg to about 7 mg of active pharmacological agent, from about 8 mg to about 12 mg of active pharmacological agent, from about 13 mg to about 19 mg of active pharmacological agent, from about 20 mg to about 30 mg of active pharmacological agent, from about 31 mg to about 60 mg of active pharmacological agent, from about 61 mg to about 80 mg of active pharmacological agent, and from about 81 mg to about 125 mg of active pharmacological agent. One preferred embodiment is a 500 mg capsule containing 100 mg of pharmacologically active agent (i.e. 500 mg of a composition of the invention containing 20% pharmacologically active agent by weight of the pharmaceutical composition).

Generally, the compositions of the invention include a first solubilizer. Generally, the solubilizer is present in an amount of from about 10% to about 50% by weight of the composition. Any suitable solubilizer known in the art can be used. Suitable solubilizers include, for example, surfactants. In some embodiments, the solubilizer is selected from polyethylene glycol 660 hydroxystearate, vitamin E polyethylene glycol succinate, and mixtures thereof. In some embodiments, the first solubilizer includes or consists of polyethylene glycol 660 hydroxystearate.

Generally, the compositions of the invention include a second solubilizer. Generally, the solubilizer is present in an amount of from about 10% to about 50% by weight of the composition. Any suitable solubilizer known in the art can be used. Suitable solubilizers include, for example, surfactants. In some embodiments, the solubilizer is selected from polyoxyl 35 castor oil, polyoxyl 40 hydrogenated castor oil, polysorbate 80, and mixtures thereof. In some embodiments, the second solubilizer includes or consists of polyoxyl 35 castor oil.

Generally, the compositions of the invention include a diluent. Generally, the diluent is present in an amount of from about 10% to about 50% by weight of the composition. Any suitable diluent and/or solvent, or combination thereof, may be used for the diluent. In some embodiments, the diluent is selected from propylene glycol monocaprylate, caprylocaproyl polyoxyglycerides, medium chain mono and diglycerides, triglycerides of caprylic/capric acid, polyethylene glycols, propylene glycol, propylene carbonate, and mixtures thereof. In some embodiments, the diluent comprises propylene glycol monocaprylate.

In some embodiments of the invention, the pharmaceutical composition comprises a carrier or excipient system comprising:

i) a first solubilizer selected from the group consisting of polyethylene glycol 660 hydroxystearate, vitamin E polyethylene glycol succinate, and mixtures thereof;

ii) a second solubilizer selected from the group consisting of polyoxyl 35 castor oil, polyoxyl 40 hydrogenated castor oil, polysorbate 80, and mixtures thereof; and

iii) a diluent selected from the group consisting of propylene glycol monocaprylate, caprylocaproyl polyoxyglycerides, medium chain monoglycerides, medium chain diglycerides, triglycerides of caprylic/capric acid, polyethylene glycols, propylene glycol, propylene carbonate, and mixtures thereof.

In some further embodiments, the carrier or excipient system comprises:
i) polyethylene glycol 660 hydroxystearate in an amount of from about 10% to about 50% by weight of the composition;
ii) polyoxyl 35 castor oil in an amount of from about 10% to about 50% by weight of the composition; and
iii) propylene glycol monocaprylate in an amount of from about 10% to about 15% by weight of the composition.

In one embodiment, the invention provides a pharmaceutical composition comprising:

a) an active pharmacological agent comprising 4-(3-{5-chloro-1-(diphenylmethyl)-2-[2-({[2-(trifluoromethyl)benzyl]sulfonyl}amino)ethyl]-1H-indol-3-yl}propyl)benzoic acid or a pharmaceutically acceptable salt thereof in an amount of about 20% by weight of the composition; and

b) a carrier or excipient system comprising:
i) polyethylene glycol 660 hydroxystearate in an amount of about 30% by weight of the composition;
ii) polyoxyl 35 castor oil in an amount of about 30% by weight of the composition; and
iii) propylene glycol monocaprylate in an amount of about 20% by weight of the composition.

In another embodiment, the invention provides a pharmaceutical composition comprising:

a) an active pharmacological agent comprising 4-(3-{5-chloro-1-(diphenylmethyl)-2-[2-({[2-(trifluoromethyl)benzyl]sulfonyl}amino)ethyl]-1H-indol-3-yl}propyl)benzoic acid or a pharmaceutically acceptable salt thereof in an amount of about 2% by weight of the composition; and

b) a carrier or excipient system comprising:
i) polyethylene glycol 660 hydroxystearate in an amount of about 36.75% by weight of the composition;
ii) polyoxyl 35 castor oil in an amount of about 36.75% by weight of the composition; and
iii) propylene glycol monocaprylate in an amount of about 24.5% by weight of the composition.

In some embodiments, the invention provides unit dosage forms comprising a pharmaceutical composition as described above, wherein the composition contains about 100 mg of the active pharmacological agent. As discussed above, other doses can be made into unit dosage forms as is well known to those of skill in the art.

Because of the liquid nature of the resulting pharmaceutical composition, unit dosage forms such as capsules are well suited for administering the pharmaceutical composition to a patient. The invention also includes methods of preparing the pharmaceutical composition for administration, particularly via a capsule unit dosage form.

In some embodiments, the invention provides a process for preparing a pharmaceutical composition as described above, comprising the steps of:
(1) mixing the first solubilizer, second solubilizer, and diluent to produce a first homogenous solution;
(2) slowly adding the pharmacologically active agent to said first homogenous solution; and
(3) mixing with sufficient heating until the pharmacologically active agent is dissolved to produce a second homogenous solution.

To facilitate the mixing and dissolution of the first and second solubilizers and the diluent, the mixture can be heated (e.g., to from about 80° C. to about 90° C., or to about 85° C.) while mixing. In some embodiments, the temperature is maintained at 85+/−5° C.

In some embodiments, the temperature is maintained at 85+/−5° C. during the addition and mixing of the pharmacologically active agent.

As discussed above, the resultant product is suited for administration via a capsule. Accordingly, the process for preparing the pharmaceutical composition may further include encapsulating at least a portion of the second homogenous solution into one or more unit dosage capsule forms. Those of skill in the art will appreciate that any suitable encapsulation technique may be used.

In some embodiments, the second homogenous solution is cooled, preferably to about 40° C., prior to encapsulation to enhance its handling and to prevent melting or dissolution of the encapsulating material.

Those of skill in the art will readily recognize that simple modification of the steps outlined above, and the relative amounts of each of the components, will result in formation of a final product of desired size, strength and composition. Accordingly, the process described above can be used to make any of the pharmaceutical compositions described herein.

In particular, the process is useful in making such pharmaceutical compositions where the pharmaceutically effective amount of the active pharmacological agent is about 0.1 to about 20% by weight of the composition.

The process is also useful in making such pharmaceutical compositions where the solubilizer of the first and second solubilizer is selected from the group consisting of polyethylene glycol 660 hydroxystearate, vitamin E polyethylene glycol succinate, polyoxyl 35 castor oil, polyoxyl 40 hydrogenated castor oil, polysorbate 80, and mixtures thereof.

The process is also useful in making such pharmaceutical compositions where the diluent is selected from the group consisting of propylene glycol monocaprylate, caprylocaproyl polyoxyglycerides, medium chain monoglycerides, medium chain diglycerides, triglycerides of caprylic/capric acid, polyethylene glycols, propylene glycol, propylene carbonate, and mixtures thereof.

The process is also useful in making such pharmaceutical compositions where the pharmaceutical composition comprises a pharmacologically active agent and a carrier or excipient system wherein:
i) the first solubilizer is selected from the group consisting of polyethylene glycol 660 hydroxystearate, vitamin E polyethylene glycol succinate, and mixtures thereof;
ii) the second solubilizer is selected from the group consisting polyoxyl 35 castor oil, polyoxyl 40 hydrogenated castor oil, polysorbate 80, and mixtures thereof; and
iii) the diluent is selected from the group consisting of propylene glycol monocaprylate, caprylocaproyl polyoxyglycerides, medium chain monoglycerides, medium chain diglycerides, triglycerides of caprylic/capric acid, polyethylene glycols, propylene glycol, propylene carbonate, and mixtures thereof.

More particularly, the process is also useful in making such pharmaceutical compositions where the pharmaceutical composition comprising a pharmacologically active agent and a carrier or excipient system comprising:
i) polyethylene glycol 660 hydroxystearate in an amount of from about 10% to about 50% by weight of the composition;
ii) polyoxyl 35 castor oil in an amount of from about 10% to about 50% by weight of the composition; and
iii) propylene glycol monocaprylate in an amount of from about 10% to about 30% by weight of the composition.

As described above, the process can be used to make various sized unit dosage forms. Generally, the dosage forms contain from about 1 mg to about 125 mg of active pharmacological agent. Typical unit dosage forms will contain about 5, 10, 25, 50, 75, 100 or 125 mg active agent. Accordingly, the invention includes dosage forms comprising a pharmaceutical composition of the invention, wherein the composition comprises from about 3 mg to about 7 mg of active pharmacological agent, from about 8 mg to about 12 mg of active pharmacological agent, from about 13 mg to about 19 mg of active pharmacological agent, from about 20 mg to about 30 mg of active pharmacological agent, from about 31 mg to about 60 mg of active pharmacological agent, from about 61 mg to about 80 mg of active pharmacological agent, and from about 81 mg to about 125 mg of active pharmacological agent. One preferred embodiment is a 500 mg capsule containing 100 mg of pharmacologically active agent (i.e. 20% by weight of the pharmaceutical composition). Another embodiment includes a 500 mg capsule containing 10 mg of pharmacologically active agent (i.e. 2% by weight of the pharmaceutical composition).

In one embodiment, the invention provides a process for preparing a preferred pharmaceutical composition comprising:

a) 20% by weight of the composition of the active pharmacological agent 4-(3-{5-chloro-1-(diphenylmethyl)-2-[2-({[2-(trifluoromethyl)benzyl]sulfonyl}amino) ethyl]-1H-indol-3-yl}propyl)benzoic acid or a pharmaceutically acceptable salt thereof; and

b) a carrier or excipient system comprising:
i) polyethylene glycol 660 hydroxystearate in an amount of from about 10% to about 50% by weight of the composition;
ii) polyoxyl 35 castor oil in an amount of from about 10% to about 50% by weight of the composition; and
iii) propylene glycol monocaprylate in an amount of from about 10% to about 30% by weight of the composition;
   said process comprising

(1) mixing the polyethylene glycol 660 hydroxystearate, polyoxyl 35 castor oil, and propylene glycol monocaprylate to produce a first homogenous solution;

(2) slowly adding the pharmacologically active agent;

(3) mixing with sufficient heating until the pharmacologically active agent is dissolved to produce a second homogenous solution.

As with the other embodiments described herein, the process can further include one or more of the additional steps of heating the polyethylene glycol 660 hydroxystearate, polyoxyl 35 castor oil, and propylene glycol monocaprylate to a temperature sufficient to produce the first homogenous solution; cooling the first homogenous solution prior to adding the pharmacologically active agent; encapsulating at least a portion of the second homogenous solution into one or more unit dosage capsule forms; and/or cooling the second homogenous solution (e.g., to about 40° C.) prior to encapsulation.

The invention further includes any product made by any of the processes described herein.

As used herein, the terms “pharmaceutically effective amount” or “therapeutically effective amount” mean the total amount of each active component of the pharmaceutical composition or method that is sufficient to show a meaningful patient benefit, i.e., treatment, healing, prevention, inhibition or amelioration of a physiological response or condition, such as an inflammatory condition or pain, or an increase in rate of treatment, healing, prevention, inhibition or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The term “pharmaceutically acceptable” means a non-toxic material that does not interfere with the effectiveness of the biological activity of the active ingredient(s).

The term “% by weight of the composition” and the weight percentages set forth for each of the components of the compositions disclosed herein refer to the percentages that each component will comprise in a final pharmaceutical composition based on the weight of the composition, excluding any surface covering, such as a tablet coating or encapsulating material, such as a capsule.

The term “caprylocaproyl polyoxyglycerides” refers to a lipid-based surface-active agent. One exemplary caprylocaproyl polyoxyglycerides is PEG-8 caprylic/capric glycerides, marketed as LABRASOL® by Gattefosse. Caprylocaproyl polyoxyglycerides are also known as “caprylocaproyl macrogolglycerides”

As used herein, the term “medium chain monoglyceride” refers to a monoacylglycerol having from about 8 to about 18 carbon atoms in the acyl chain.

As used herein, “a medium chain diglyceride” refers to a diacylglycerol having, independently, from about 8 to about 18 carbon atoms in each acyl chain.

As will be appreciated, some components of the formulations of the invention can possess multiple functions. For example, a given component can act as both a diluent and a solubilizer. In some such cases, the function of a given component can be considered singular, even though its properties may allow multiple functionality.

The pharmaceutical formulations and excipient systems herein can also contain an antioxidant or a mixture of antioxidants, such as ascorbic acid. Other antioxidants, which can be used, include sodium ascorbate and ascorbyl palmitate, optionally in conjunction with an amount of ascorbic acid. An example range for the antioxidant(s) is from about up to about 15% by weight, e.g., from about 0.05% to about 15% by weight, from about 0.5% to about 15% by weight, or from about 0.5% to about 5% by weight. In some embodiments, the pharmaceutical formulations contain substantially no antioxidant.

Additional numerous various viscosity builders, surfactant/solubilizers, diluents/solvents, dispersing agents, excipients, dosage forms, and the like, that are suitable for use in connection with the pharmaceutical compositions of the invention are known in the art and described in, for example, Remington: The Science and Practice of Pharmacy, 20th edition, Alfonoso R. Gennaro (ed.), Lippincott Williams & Wilkins, Baltimore, Md. (2000), which is incorporated herein by reference in its entirety.

The materials, methods, and examples presented herein are intended to be illustrative, and are not intended to limit the scope of the invention. All publications, patent applications, patents, and other references mentioned herein are incorporated by reference in their entirety.

### EXAMPLES

### 1. Preparation of compounds of Formula I or Formula II

The compounds of Formula I or Formula II can be conveniently prepared from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but one skilled in the art can determine such conditions by routine optimization procedures. Those skilled in the art will recognize that the nature and order of the synthetic steps presented may be varied for the purpose of optimizing the formation of the compounds of the invention.

Preparation of compounds of Formula I or Formula II can involve the protection and deprotection of various chemical groups. The need for protection and deprotection, and the selection of appropriate protecting groups can be readily determined by one skilled in the art. The chemistry of protecting groups can be found, for example, in Greene, et al., Protective Groups in Organic Synthesis, 4th Ed., Wiley & Sons, 2006, which is incorporated herein by reference in its entirety.

Examples of compounds of Formula I or Formula II and methods for synthesizing them can be found in U.S. Pat. Nos. 6,797,708; 6,891,065 and 6,984,735 and U.S. patent application Ser. Nos. 10/930,534 (filed Aug. 31, 2004), 10/948,004 (filed Sep. 23, 2004), 10/989,840 (filed Nov. 16, 2004), 11/014,657 (filed Dec. 16, 2004), 11/064,241 (filed Feb. 23, 2005), 11/088,568 (filed Mar. 24, 2005), 11/140,390 (filed May 27, 2005), 11/207,072 (filed Aug. 18, 2005) and 11/442,199 (filed May 26, 2006), each of which is incorporated by reference in their entireties.

Examples of compounds of Formula I and Formula II include, but are not limited to:

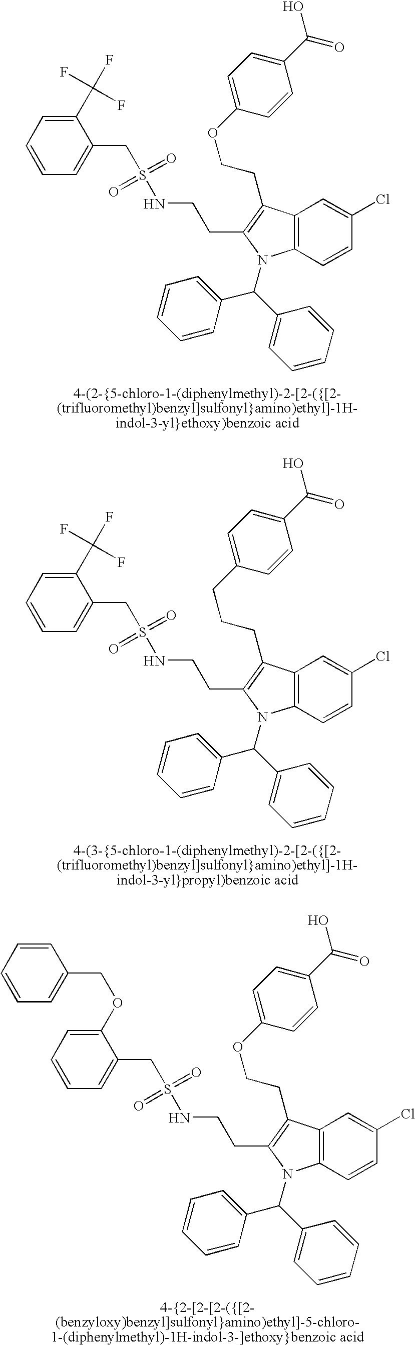

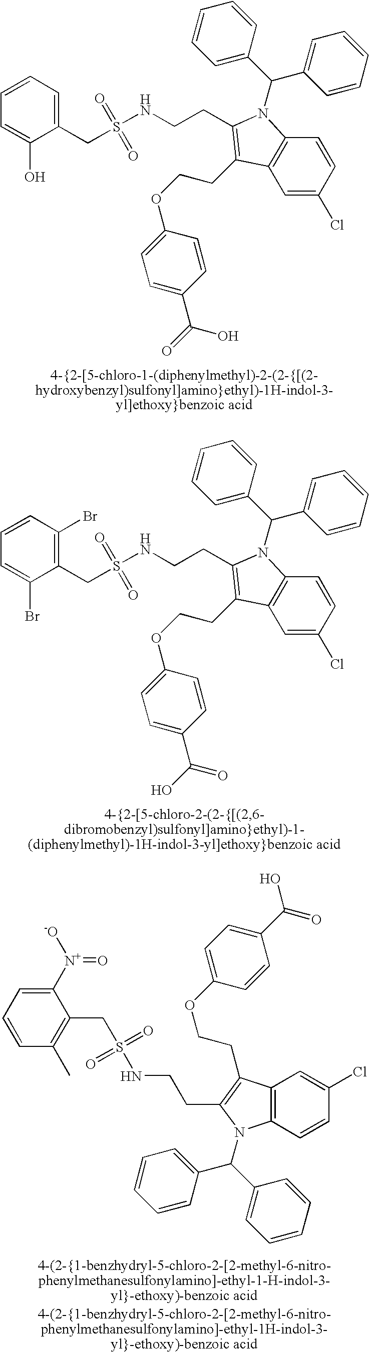

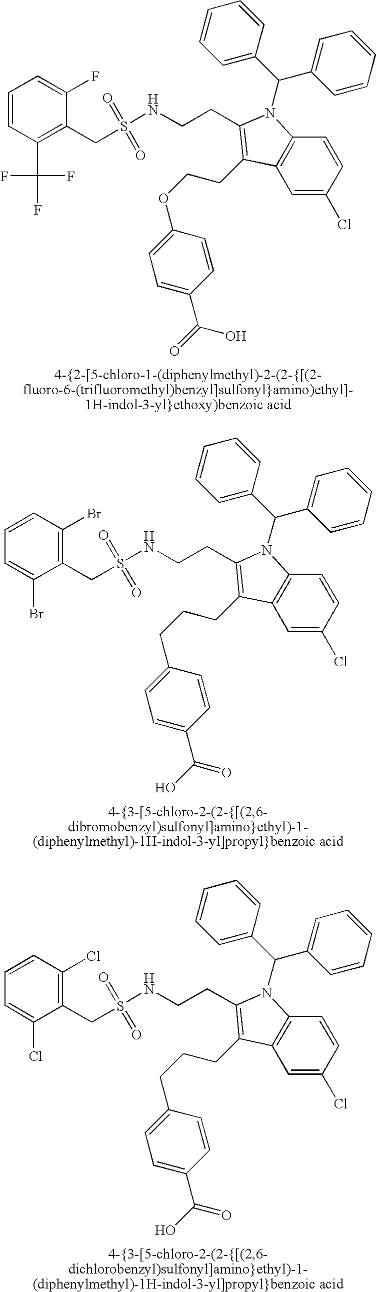

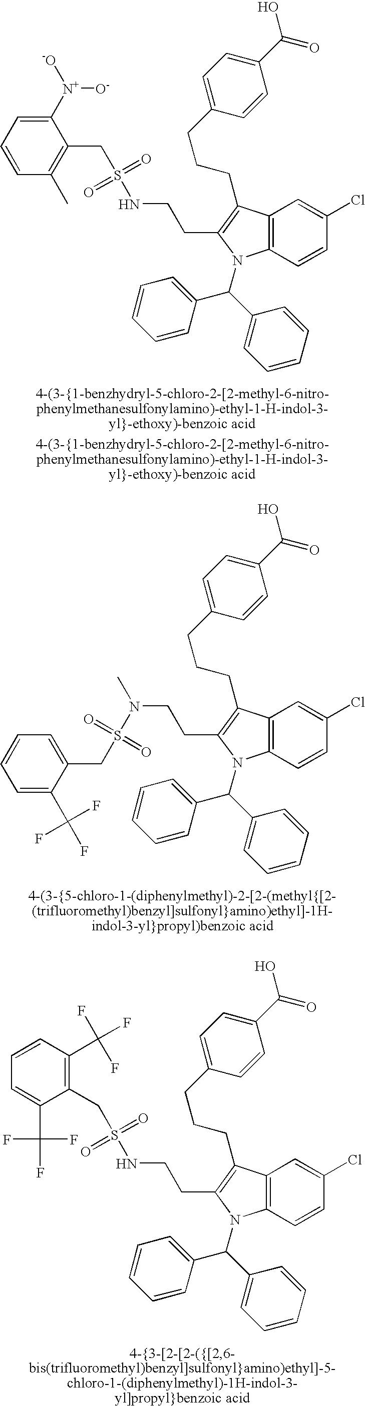

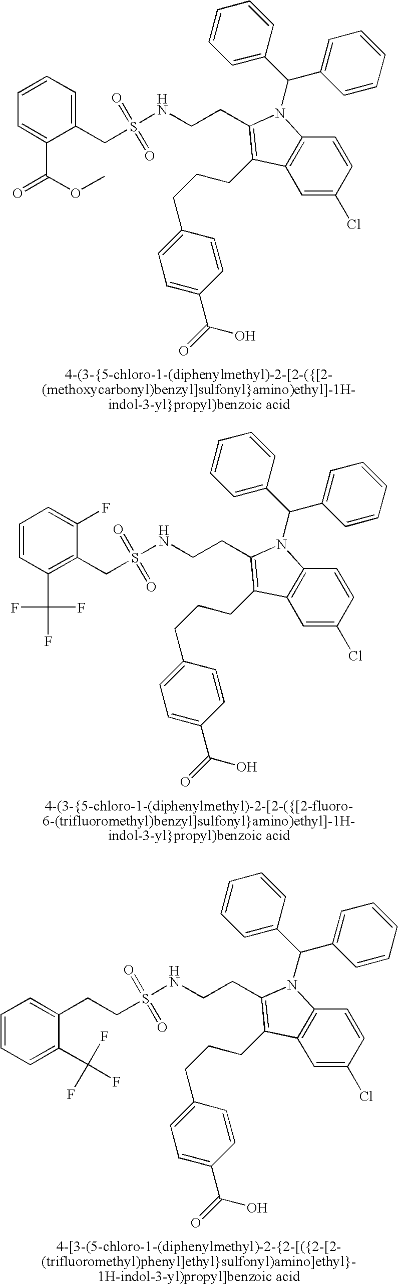

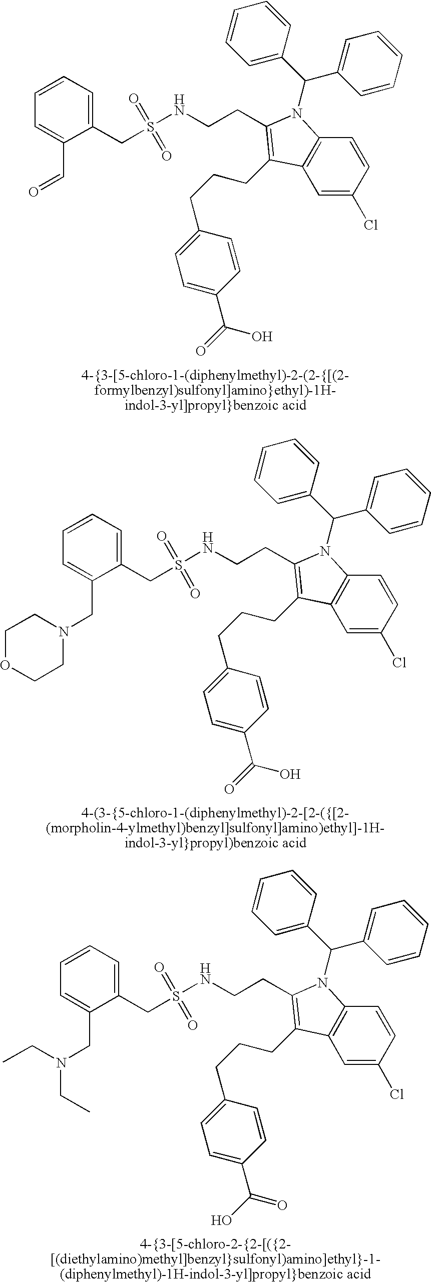

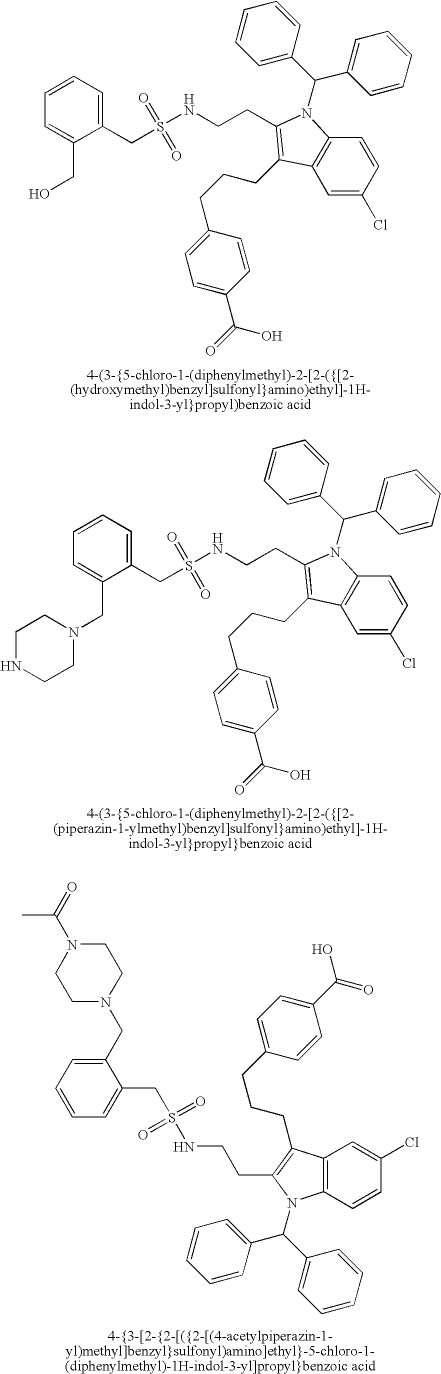

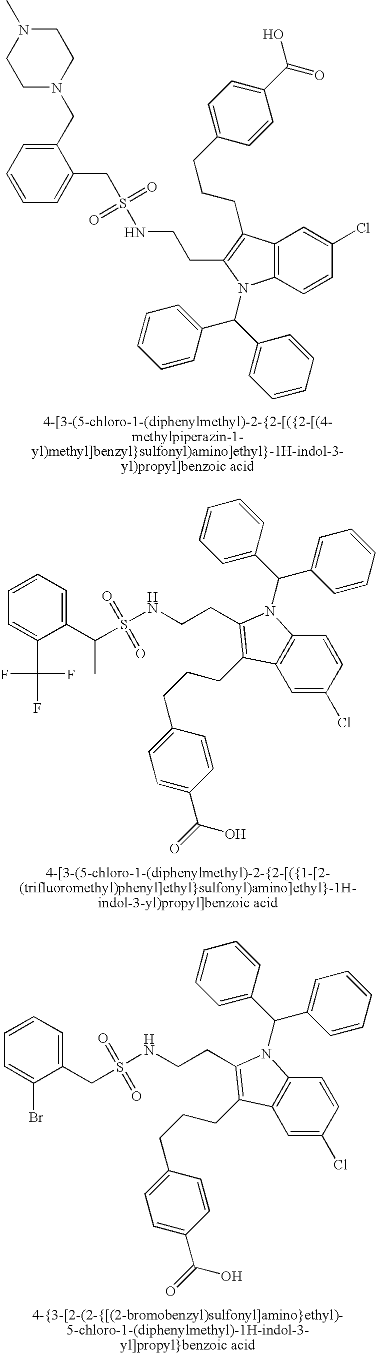

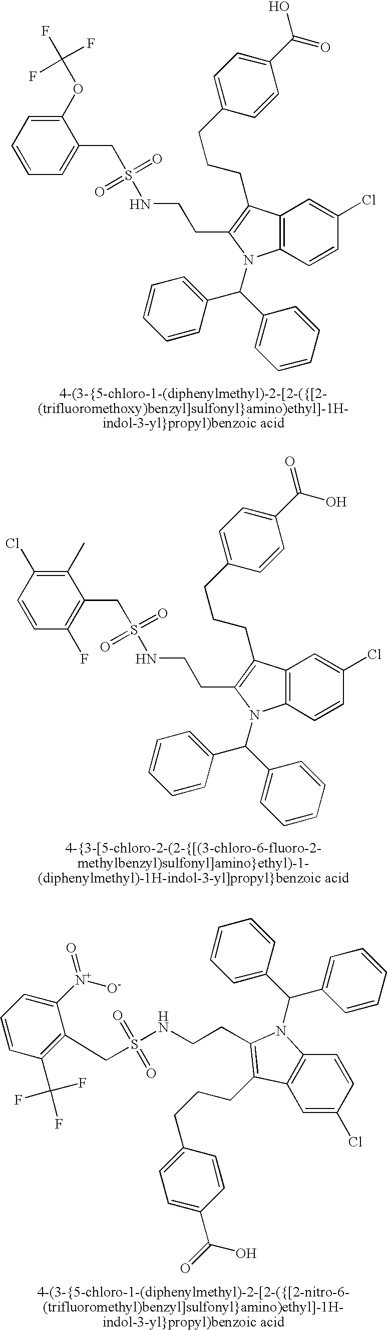

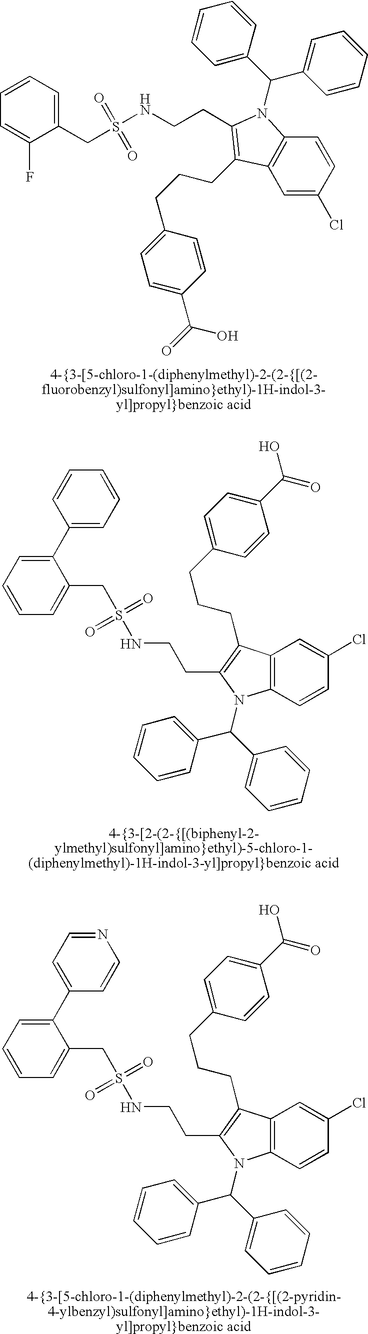

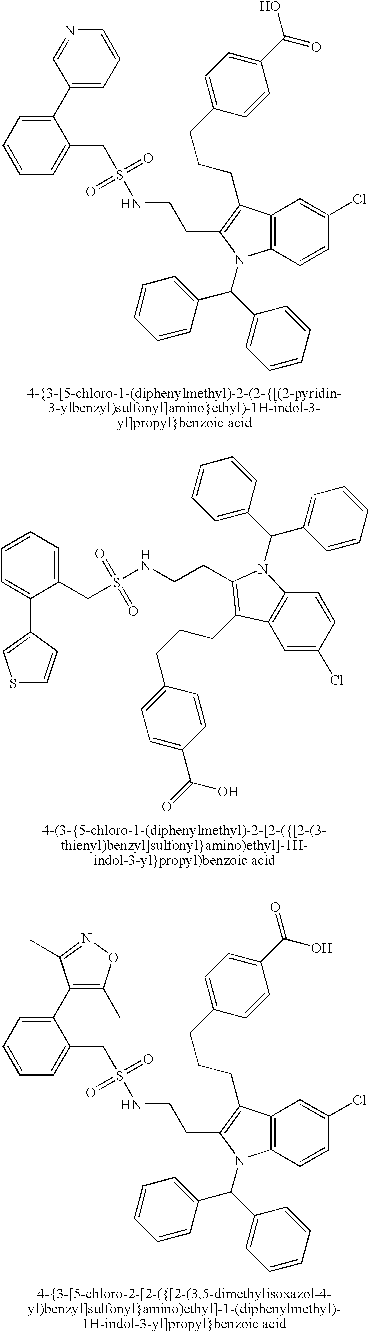

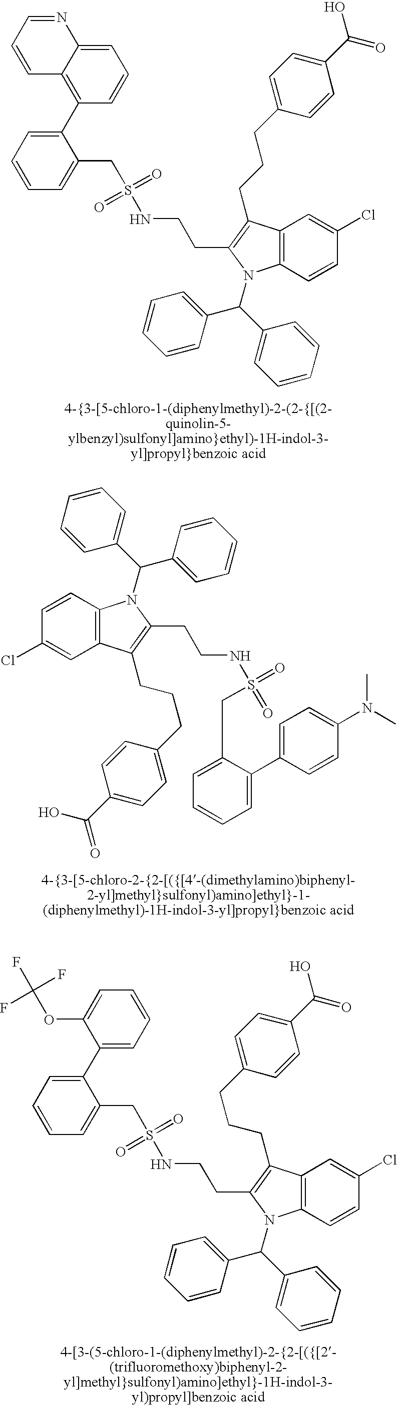

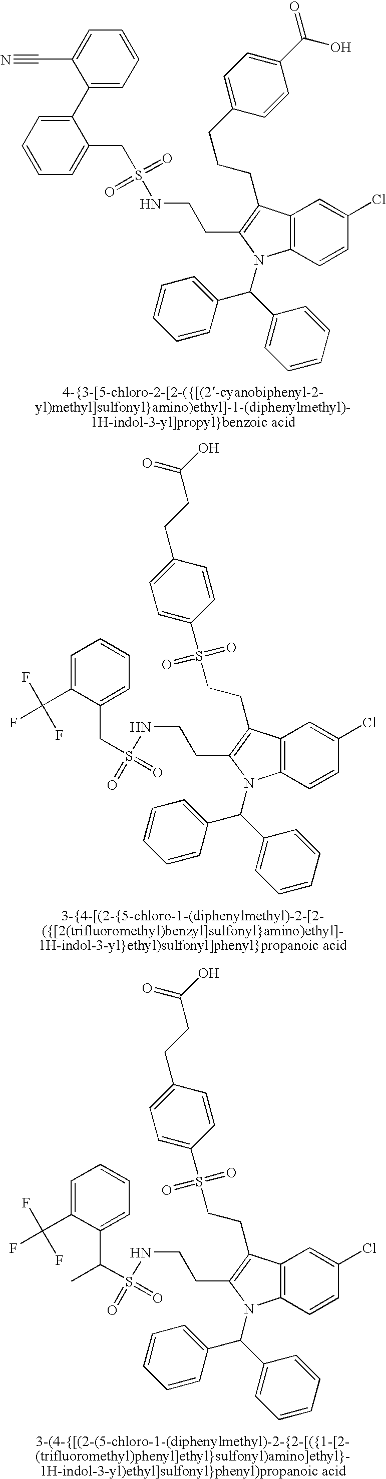

### 2. Formulations containing 4-(3-{5-chloro-1-(diphenylmethyl)-2-[2-({[2-(trifluoromethyl)benzyl]sulfonyl}amino)ethyl]-1H-indol-3-yl}propyl)benzoic acid

### A. Preparation of 100 mg dose capsule

A 500 mg unit dosage capsule in accordance with the invention, containing a 100 mg dose of 4-(3-{5-chloro-1-(diphenylmethyl)-2-[2-({[2-(trifluoromethyl)benzyl]sulfonyl}amino)ethyl]-1H-indol-3-yl}propyl)benzoic acid was prepared as described in Table 1.

TABLE 1

| | | % Wt of | Weight |
|---|---|---|---|
| Component | Compound | Composition | (mg) |
| Pharmacological | 4-(3-{5-chloro-1-(diphenylmethyl)-2-[2-({[2- | 20 | 100 |
| Agent | (trifluoromethyl)benzyl]sulfonyl}amino)ethyl]- | | |
| | 1H-indol-3-yl}propyl)benzoic acid | | |
| First Solubilizer | polyethylene glycol 660 hydroxystearate | 30 | 150 |
| Second | polyoxyl 35 castor oil | 30 | 150 |
| Solubilizer | | | |
| Diluent | propylene glycol monocaprylate | 20 | 100 |

The pharmaceutical composition described above was prepared for administration via a capsule as follows:
1. Polyethylene glycol 660 hydroxystearate (30 g), polyoxyl 35 castor oil (30 g), and propylene glycol monocaprylate (20 g) were added into an appropriate mixing vessel equipped for temperature control.
2. The vessel was heated to 85+/−5° C. with mixing until a homogeneous solution was obtained.
3. The pharmacological agent (20 g) was added slowly into the solution in Step 2, with heating and mixing at 85+/−5° C. until the drug was dissolved and a homogeneous solution was obtained.
4. 0.500 g of the finished solution from Step 3 was encapsulated into size #0 capsules.

Any suitable encapsulating techniques and apparatus may be used. The resultant capsule is approximately a 500 mg capsule, which delivers approximately 100 mg of the pharmacological agent. Other suitable doses and capsule sizes can be made in accordance with the disclosure herein. In particular, those of skill in the art, will readily recognize that 10, 25, 50, 75, 100 and 125 mg unit dosage forms, and others, can be made through similar methods.

### B. Dissolution Testing

The solubility of 4-(3-{5-chloro-1-(diphenylmethyl)-2-[2-({[2-(trifluoromethyl)benzyl]sulfonyl}amino)ethyl]-1H-indol-3-yl}propyl)benzoic acid was measured at room temperature in water, acid and basic conditions. The intrinsic solubility of the free acid was below the HPLC detection limit of 31 ng/mL, whereas the anion had a solubility of 110 ng/mL.

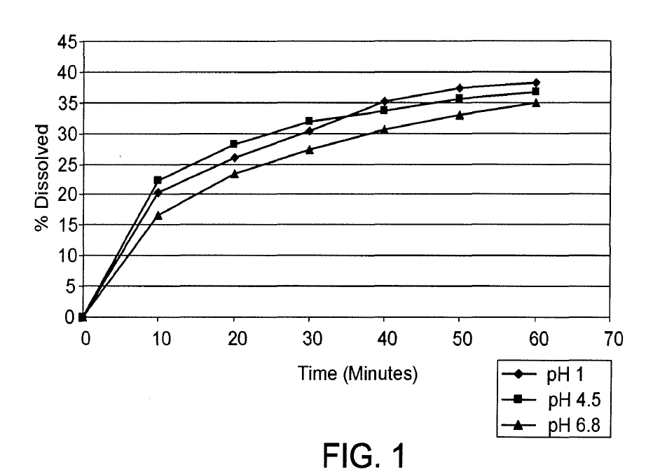

Dissolution testing was performed on 100 mg strength capsules produced according to the procedure described above. Capsules were placed in 900 mL of aqueous solutions having pH 1 (0.1 N HCl), pH 6.8 (50 mM sodium phosphate buffer) and pH 4.5 (mM sodium acetate buffer). The UV absorption of each solution was measured at various timepoints (1 mm path length, 237 nm) and the percent dissolution was calculated compared to a standard response at that wavelength. As shown in FIG. 1, the rate of dissolution was found to be similar at each pH tested.

### C. In Vivo Dog Exposure Studies

A formulation containing 4-(3-{5-chloro-1-(diphenylmethyl)-2-[2-({[2-(trifluoromethyl)benzyl]sulfonyl}amino)ethyl]-1H-indol-3-yl}propyl)benzoic acid according to the invention was studied in dogs in a high fat-fed/fasted study at approximately 12 mg/kg. To simulate the fed state, three female beagle dogs were fed a high-fat diet by oral gavage 30 minutes prior to dosing with 100 mg dose capsules as described in Table 1 above. Blood samples were drawn at 0, 0.5, 1, 2, 3, 4, 6, 8, 12 and 24 hours. The dogs were then fed ⅔ of the daily food ration after the 4 hour blood draw. Blood samples were stored on ice, centrifuged at 5° C., and the plasma was collected and stored at −70° C. The plasma samples were analyzed by LC/MS/MS to determine the amount of 4-(3-{5-chloro-1-(diphenylmethyl)-2-[2-({[2-(trifluoromethyl)benzyl]sulfonyl}amino)ethyl]-1H-indol-3-yl}propyl)benzoic acid in the sample.

To simulate the fasted state, the above procedure was repeated with the same three female beagle dogs that were fasted overnight prior to dosing, then fed after the 4 hour blood draw. The results of both the fed and fasted studies are summarized in Table 2 (reported results are the average of the data from the three test animals).

TABLE 2

| | Cₘₐₓ | AUC_{inf} | | | % Bio- | Fed/Fasted | Fed/Fasted |
|---|---|---|---|---|---|---|---|
| Formulation | (ng/mL) | (ng hr/mL) | AUC/Dose | Cₘₐₓ/Dose | availability | AUC/Dose | Cₘₐₓ/Dose |
| Fasted | 2873 | 17144 | 1593 | 266.2 | 8.38 | 2.14 | 1.53 |
| Fed | 4316 | 36239 | 3471 | 411.7 | 18.27 | | |

Data from a rat carrageenan-induced paw edema (CPE) study indicated the minimum efficacious exposure of 4-(3-{5-chloro-1-(diphenylmethyl)-2-[2-({[2-(trifluoromethyl)benzyl]sulfonyl}amino)ethyl]-1H-indol-3-yl}propyl)benzoic acid was 1360 ng*hr/ml. The data in Table 2 shows that the formulation according to the present invention results in an exposure of about 12.5 times the efficacious exposure in the fasted state and about 26.6 times the efficacious exposure in the fed state. These exposures translate into percent bioavailabilities of 8.4 and 18.3 when compared to an IV formulation (15% 4-(3-{5-chloro-1-(diphenylmethyl)-2-[2-({[2-(trifluoromethyl)benzyl]sulfonyl}amino)ethyl]-1H-indol-3-yl}propyl)benzoic acid, 10% EtOH, 75% Solutol HS-15, diluted to 2 mg/mL with sterile water for injection).

### 3. Formulations containing 4-(3-{5-chloro-1-(diphenylmethyl)-2-[2-({[2-(trifluoromethoxy)benzyl]sulfonyl}amino)ethyl]-1H-indol-3-yl}propyl)benzoic acid

The solubility of 4-(3-{5-chloro-1-(diphenylmethyl)-2-[2-({[2-(trifluoromethoxy)benzyl]sulfonyl}amino)ethyl]-1H-indol-3-yl}propyl)benzoic acid was measured at room temperature in water, acid and basic conditions. The intrinsic solubility in all conditions is below the HPLC detection limit of 21.2 ng/mL.

Due to the low solubility of 4-(3-{5-chloro-1-(diphenylmethyl)-2-[2-({[2-(trifluoromethoxy)benzyl]sulfonyl}amino)ethyl]-1H-indol-3-yl}propyl)benzoic acid in a 2% Tween 80/0.5% methylcellulose (MC) vehicle (0.496 mg/ml), alternative formulations having enhanced dissolution/solubility properties were explored. The addition of 2% Tween 80 enhancing the 4-(3-{5-chloro-1-(diphenylmethyl)-2-[2-({[2-(trifluoromethoxy)benzyl]sulfonyl}amino)ethyl]-1H-indol-3-yl}propyl)benzoic acid solubility 23.490-fold to 0.498 mg/ml, did not afford adequate oral exposure. Following a single oral dose of 25 mg/kg of 4-(3-{5-chloro-1-(diphenylmethyl)-2-[2-({[2-(trifluoromethoxy)benzyl]sulfonyl}amino)ethyl]-1H-indol-3-yl}propyl)benzoic acid in 2% Tween/0.5% MC, the oral absorption of 4-(3-{5-chloro-1-(diphenylmethyl)-2-[2-({[2-(trifluoromethoxy)benzyl]sulfonyl}amino)ethyl]-1H-indol-3-yl}propyl)benzoic acid in rats was found to be relatively low, resulting in an estimated bioavailability of only about 1.8%. A liquid formulation containing 20% 4-(3-{5-chloro-1-(diphenylmethyl)-2-[2-({[2-(trifluoromethoxy)benzyl]sulfonyl}amino)ethyl]-1H-indol-3-yl}propyl)benzoic acid, 30% Cremophor EL, 30% Solutol HS-15 and 20% Capryol 90 (CESC) was found to provide a faster absorption rate and increased bioavailability (about 9.7%) in non-fasted rats at 25 mg/kg. Based on the animal data and solubility in pharmaceutical acceptable excipients, formulation development for first in human studies was undertaken based on this formulation.

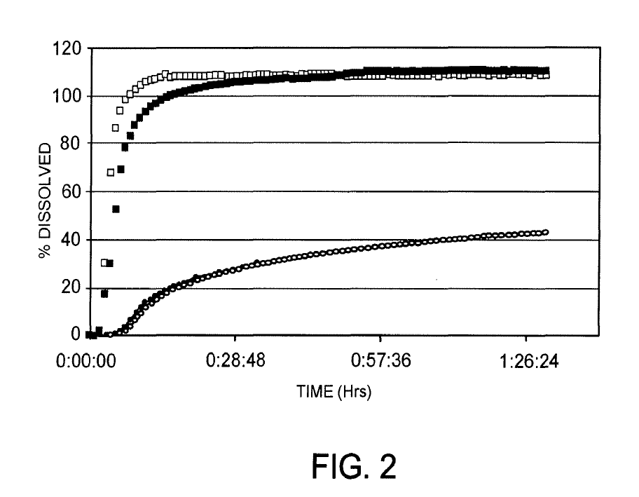

A prototype CESC capsule formulation, batch size of 400 g, was manufactured according to methods similar to those described above in Example 2. The dissolution profiles of the CESC liquid capsule formulation at the 100 mg strength and encapsulated micronized 4-(3-{5-chloro-1-(diphenylmethyl)-2-[2-({[2-(trifluoromethoxy)benzyl]sulfonyl}amino)ethyl]-1H-indol-3-yl}propyl)benzoic acid are shown in FIG. 2. These dissolution profiles were obtained in a medium containing 0.3% sodium lauryl sulfate (SLS)/50 mM phosphate pH 7.5 buffer. As shown in FIG. 2, the CESC liquid formulation was found to significantly improve dissolution of the 4-(3-{5-chloro-1-(diphenylmethyl)-2-[2-({[2-(trifluoromethoxy)benzyl]sulfonyl}amino)ethyl]-1H-indol-3-yl}propyl)benzoic acid.

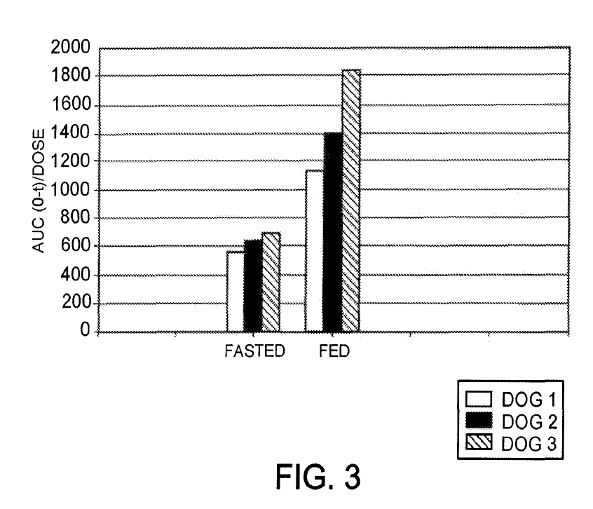

This CESC formulation was compared to five other prototype formulations and was screened in dogs in a high fat-fed/fasted study at 10 mpk using capsule strengths of 100 mg. The results show that the CESC formulation shows less inter-subject variability then the other formulations (see FIG. 3).

Since the minimum efficacious exposure is 2800 ng·hr/ml (ApoE Mice), the data in Table 3 shows an exposure of 2.1 x's the efficacious exposure in the fasted state and 4.5 x's in the fed state. This translates into % bioavailabilities of 3.1 (fasted) and 6.8 (fed) when comparing to an IV formulation.

TABLE 3
Fed/Fasted Dog Study 100 mg Capsule per Dog

| | | | | AUC (0- |
|---|---|---|---|---|
| Condition | Cmax (ng/ml) | AUC (0-Inf) | Cmax/Dose | Inf)/Dose |
| Fasted | 926 (232) | 5803 | 108 (29.4) | 632 (65) |
| Fed | 2474 (885) | 12610 (1328) | 281 (82) | 1457 (361) |

Capsules containing 10 mg, 25 mg, and 100 mg of 4-(3-{5-chloro-1-(diphenylmethyl)-2-[2-({[2-(trifluoromethoxy)benzyl]sulfonyl}amino)ethyl]-1H-indol-3-yl}propyl)benzoic acid were prepared according to Table 4 and encapsulated at a capsule fill weight of 50 mg, 125 mg and 500 mg, respectively. Formulation compositions in all strengths are the same and the only difference is fill weight. The formulation was filled into size #0 grey Licaps (hard gelatin) capsules.

TABLE 4

| | | | Amount (mg) | | |
|---|---|---|---|---|---|
| | | % | 10 mg | 25 mg | 100 mg |
| Compound | Component | Wt/Wt | capsule | capsule | capsule |
| 4-(3-{5-chloro-1- | active | 20 | 10 | 25 | 100 |
| (diphenylmethyl)-2-[2- | pharmacological | | | | |
| ({[2- | agent | | | | |
| (trifluoromethoxy)benzyl]- | | | | | |
| sulfonyl}amino)ethyl]- | | | | | |
| 1H-indol-3-yl}propyl)benzoic | | | | | |
| acid | | | | | |
| Macrogol-15- | First | 30 | 15 | 37.5 | 150 |
| Hydroxystearate (Solutol | Solubilizer | | | | |
| HS 15) | | | | | |
| Polyoxyl 35 Castor Oil | Second | 30 | 15 | 37.5 | 150 |
| (Cremophor EL) | solubilizer | | | | |
| Propylene Glycol | Diluent | 20 | 10 | 25 | 100 |
| Monocaprylate 90% | | | | | |
| (Capryol 90)^{a} | | | | | |
| Total | | 100 | 50 | 125 | 500 |

### 4. Formulations containing 4-(3-{5-chloro-1-(diphenylmethyl)-2-[2-({[2-fluoro-6-(trifluoromethyl)benzyl]sulfonyl}amino)ethyl]-1H-indol-3-yl}propyl)benzoic acid

The solubility of 4-(3-{5-chloro-1-(diphenylmethyl)-2-[2-({[2-fluoro-6-(trifluoromethyl)benzyl]sulfonyl}amino)ethyl]-1H-indol-3-yl}propyl)benzoic acid was measured at room temperature in water, acid and basic conditions. The intrinsic solubility over the pH range of 1 to 11 is below the HPLC detection limit of 100 ng/mL.

Due to the low solubility of 4-(3-{5-chloro-1-(diphenylmethyl)-2-[2-({[2-fluoro-6-(trifluoromethyl)benzyl]sulfonyl}amino)ethyl]-1H-indol-3-yl}propyl)benzoic acid in a 2% Tween 80/0.5% methylcellulose vehicle, (0.115 mg/ml), alternative formulations having enhanced dissolution/solubility properties were explored. The addition of 2% Tween 80 enhancing the PLA-811 solubility, did not afford adequate oral exposure. Following a single oral dose of 25 mg/kg of 4-(3-{5-chloro-1-(diphenylmethyl)-2-[2-({[2-fluoro-6-(trifluoromethyl)benzyl]sulfonyl}amino)ethyl]-1H-indol-3-yl}propyl)benzoic acid in 2% Tween/0.5% MC, the oral absorption of 4-(3-{5-chloro-1-(diphenylmethyl)-2-[2-({[2-fluoro-6-(trifluoromethyl)benzyl]sulfonyl}amino)ethyl]-1H-indol-3-yl}propyl)benzoic acid in rats was found to be relatively low, resulting in an estimated bioavailability of only <1%.

A prototype CESC capsule formulation, batch size of 10 g, was manufactured according to methods similar to those described above in Example 2. A 500 mg capsule was prepared according to Table 5.

TABLE 5

| | | | Amount |
|---|---|---|---|
| | | | (mg) |
| | | % | 100 mg |
| Compound | Component | Wt/Wt | capsule |
| 4-(3-{5-chloro-1-(diphenylmethyl)- | active | 20 | 100 |
| 2-[2-({[2-fluoro-6- | pharmaco- | | |
| (trifluoromethyl)benzyl]sulfonyl} | logical | | |
| amino)ethyl]-1H-indol-3- | agent | | |
| yl}propyl)benzoic acid | | | |
| Macrogol-15-Hydroxystearate | First | 30 | 150 |
| (Solutol HS 15) | Solubilizer | | |
| Polyoxyl 35 Castor Oil | Second | 30 | 150 |
| (Cremophor EL) | solubilizer | | |
| Propylene Glycol Monocaprylate 90% | Diluent | 20 | 100 |
| (Capryol 90)^{a} | | | |
| Total | | 100 | 500 |

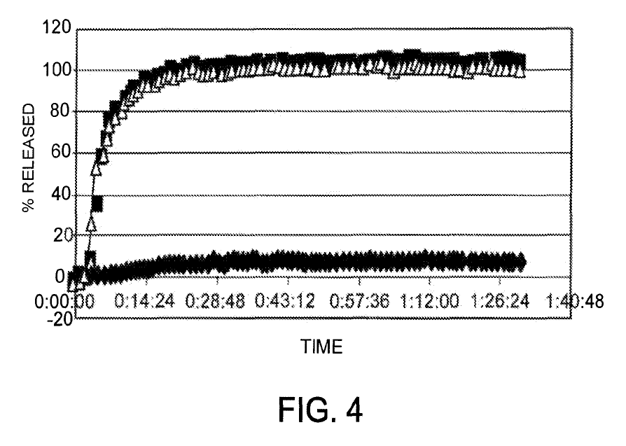

The dissolution profiles of the CESC liquid capsule formulation at the 100 mg strength and encapsulated, micronized 4-(3-{5-chloro-1-(diphenylmethyl)-2-[2-({[2-fluoro-6-(trifluoromethyl)benzyl]sulfonyl}amino)ethyl]-1H-indol-3-yl}propyl)benzoic acid are presented in FIG. 4. The dissolution profiles were obtained in a medium containing 0.3% SLS/50 mM phosphate pH 7.5 buffer. As shown in FIG. 4, the CESC liquid formulation was found to significantly improve dissolution of 4-(3-{5-chloro-1-(diphenylmethyl)-2-[2-({[2-fluoro-6-(trifluoromethyl)benzyl]sulfonyl}amino)ethyl]-1H-indol-3-yl}propyl)benzoic acid.

All publications mentioned herein, including but not limited to patent applications, patents, and other references, are incorporated by reference in their entirety.

The materials, methods, and examples presented herein are intended to be illustrative, and are not intended to limit the scope of the invention.

1. A pharmaceutical composition comprising
a) a pharmaceutically effective amount of an active pharmacological agent having Formula I: 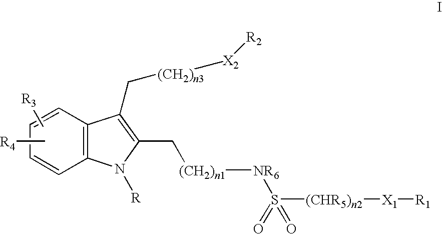
or a pharmaceutically acceptable salt thereof, wherein:
R is —(CH₂)ₙ-A, wherein A is: 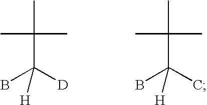
wherein
B and C are each phenyl, each independently optionally substituted by from 1 to 3 substituents selected independently from halogen, —CN, —CHO, —CF₃, —OCF₃, —OH, C₁-C₆ alkyl, C₁-C₆ alkoxy, —NH₂, —N(C₁-C₆ alkyl)₂, —NH(C₁-C₆ alkyl), —NH—C(O)—(C₁-C₆ alkyl), and —NO₂;
n is an integer from 0 to 3;
n₁ is an integer from 1 to 3;
n₂ is an integer from 0 to 4;
n₃ is an integer from 0 to 3;
n₄ is an integer from 0 to 2;
X₁ is selected from a chemical bond, —S—, —O—, —S(O)—, —S(O)₂—, —NH—, —C═C—, 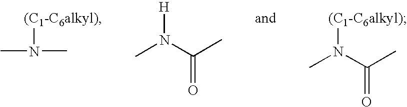
R₁ is selected from C₁-C₆ alkyl, C₁-C₆ fluorinated alkyl, C₃-C₆ cycloalkyl, tetrahydropyranyl, camphoryl, adamantyl, CN, —N(C₁-C₆ alkyl)₂, phenyl, pyridinyl, pyrimidinyl, furyl, thienyl, naphthyl, morpholinyl, triazolyl, pyrazolyl, piperidinyl, pyrrolidinyl, imidazolyl, piperizinyl, thiazolidinyl, thiomorpholinyl, tetrazolyl, indolyl, benzoxazolyl, benzofuranyl, imidazolidine-2-thionyl, 7,7-dimethyl-bicyclo[2.2.1]heptan-2-onyl, benzo[1,2,5]oxadiazolyl, 2-oxa-5-aza-bicyclo[2.2.1]heptanyl, piperazin-2-onyl and pyrrolyl groups, each optionally substituted by from 1 to 3 substituents independently selected from halogen, —CN, —CHO, —CF₃, —OCF₃, —OH, C₁-C₆ alkyl, C₁-C₆ alkoxy, —NH₂, —N(C₁-C₆ alkyl)₂, —NH(C₁-C₆ alkyl), —NH—C(O)—(C₁-C₆ alkyl), —NO₂, —SO₂(C₁-C₃ alkyl), —SO₂NH₂, —SO₂NH(C₁-C₃ alkyl), —SO₂N(C₁-C₃ alkyl)₂, —COOH, —CH₂—COOH, —CH₂—NH(C₁-C₆ alkyl), —CH₂—N(C₁-C₆ alkyl)₂, —CH₂—NH₂, pyridinyl, 2-methyl-thiazolyl, morpholino, 1-chloro-2-methyl-propyl, C₁-C₆thioalkyl, phenyl (further optionally substituted with one or more halogens, dialkylamino, —CN, or —OCF₃), benzyloxy, —(C₁-C₃ alkyl)C(O)CH₃, —(C₁-C₃ alkyl)OCH₃, —C(O)NH₂, or 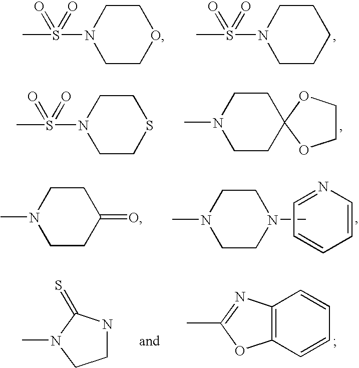
X₂ is selected from —O—, —CH₂—, —S—, —SO—, —SO₂—, —NH—, —C(O)—, 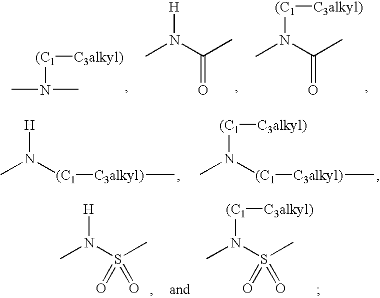
R₂ is phenyl, substituted by a group of the formula —(CH₂)ₙ₄—CO₂H or a pharmaceutically acceptable acid mimic or mimetic; and also optionally substituted by 1 or 2 additional substituents independently selected from halogen, —CN, —CHO, —CF₃, —OCF₃, —OH, C₁-C₆ alkyl, C₁-C₆ alkoxy, C₁-C₆ thioalkyl, —NH₂, —N(C₁-C₆ alkyl)₂, —NH(C₁-C₆ alkyl), —NH—C(O)—(C₁-C₆ alkyl), and —NO₂;
R₃ is selected from H, halogen, —CN, —CHO, —CF₃, —OCF₃, —OH, C₁-C₆ alkyl, C₁-C₆ alkoxy, C₁-C₆ thioalkyl, —NH₂, —N(C₁-C₆ alkyl)₂, —NH(C₁-C₆ alkyl), —NH—C(O)—(C₁-C₆ alkyl), and —NO₂;
R₄ is selected from H, halogen, —CN, —CHO, —CF₃, —OCF₃, —OH, C₁-C₆ alkyl, C₁-C₆ alkoxy, C₁-C₆ thioalkyl, —NH₂, —N(C₁-C₆ alkyl)₂, —NH(C₁-C₆ alkyl), —NH—C(O)—(C₁-C₆ alkyl), —NO₂, morpholino, pyrrolidino, piperidinyl, and piperizinyl;
each R₅ is independently H or C₁₋₃ alkyl; and
R₆ is H or C₁₋₆ alkyl; and
b) a carrier or excipient system comprising:
i) about 10 to about 50% a first solubilizer by weight of the composition;
ii) about 10 to about 50% a second solubilizer by weight of the composition; and
iii) about 10 to about 30% a diluent by weight of the composition.

2. The pharmaceutical composition of claim 1, wherein
R₁ is optionally substituted phenyl; and
B and C are each unsubstituted phenyl.

3. The pharmaceutical composition of claim 1, wherein said pharmaceutically effective amount of said active pharmacological agent is about 0.1 to about 25% by weight of the composition.

4. The pharmaceutical composition of claim 1, wherein said first solubilizer is selected from the group consisting of polyethylene glycol 660 hydroxystearate, Vitamin E polyethylene glycol succinate, and mixtures thereof.

5. The pharmaceutical composition of claim 1, wherein said first solubilizer comprises polyethylene glycol 660 hydroxystearate.

6. The pharmaceutical composition of claim 1 wherein said second solubilizer is selected from the group consisting of polyoxyl 35 castor oil, polyoxyl 40 hydrogenated castor oil, polysorbate 80, and mixtures thereof.

7. The pharmaceutical composition of claim 1, wherein said second solubilizer comprises polyoxyl 35 castor oil.

8. The pharmaceutical composition of claim 1, wherein said diluent is selected from the group consisting of propylene glycol monocaprylate, a caprylocaproyl polyoxyglyceride, a medium chain monoglyceride, a medium chain diglyceride, a triglyceride of caprylic acid, a triglyceride of capric acid, a polyethylene glycol, propylene glycol, propylene carbonate, and mixtures thereof.

9. The pharmaceutical composition of claim 1, wherein said diluent comprises propylene glycol monocaprylate.

10. The pharmaceutical composition of claim 1 wherein said carrier or excipient system comprises:
i) a first solubilizer selected from the group consisting of polyethylene glycol 660 hydroxystearate, Vitamin E polyethylene glycol succinate, and mixtures thereof,
ii) a second solubilizer selected from the group consisting of polyoxyl 35 Castor oil, polyoxyl 40 hydrogenated castor oil, polysorbate 80, and mixtures thereof, and
iii) a diluent selected from the group consisting of propylene glycol monocaprylate, a caprylocaproyl polyoxyglyceride, a medium chain monoglyceride, a medium chain diglyceride a triglyceride of caprylic acid, a triglyceride of capric acid, a polyethylene glycol, propylene glycol, propylene carbonate, and mixtures thereof.

11. The pharmaceutical composition of claim 1 wherein said carrier or excipient system comprises:
i) about 10 to about 50% polyethylene glycol 660 hydroxystearate by weight of the composition;
ii) about 10 to about 50% polyoxyl 35 castor oil by weight of the composition; and
iii) about 10 to about 30% propylene glycol monocaprylate by weight of the composition.

12. A pharmaceutical composition comprising:
a) a pharmaceutically effective amount of an active pharmacological agent having the Formula II: 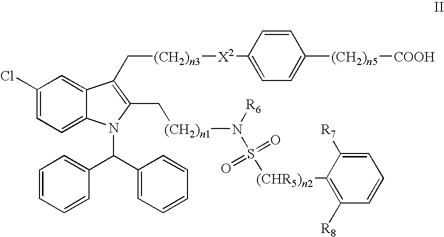
or a pharmaceutically acceptable salt thereof, wherein:
n₁ is 1 or 2;
n₂ is 1 or 2;
n₃ is 1 or 2;
n₅ is 0, 1 or 2;
X² is O, —CH₂— or SO₂;
each R₅ is independently H or C₁₋₃ alkyl;
R₆ is H or C₁₋₆ alkyl;
R₇ is selected from the group consisting of —OH, benzyloxy, —CH₃, —CF₃, —OCF₃, C₁₋₃ alkoxy, halogen, —CHO, —CO(C₁₋₃ alkyl), —CO(OC₁₋₃ alkyl), quinoline-5-yl, 3,5-dimethylisoxazol-4-yl, thiophene-3-yl, pyridin-4-yl, pyridine-3-yl, —CH₂-Q, and phenyl optionally substituted by from one to three independently selected R₃₀ groups;
R₈ is selected from the group consisting of H, —OH, —NO₂, —CF₃, —OCF₃, C₁₋₃ alkoxy, halogen, —CO(C₁₋₃ alkyl), —CO(OC₁₋₃ alkyl), quinoline-5-yl, 3,5-dimethylisoxazol-4-yl, thiophene-3-yl, —CH₂-Q, and phenyl substituted by from one to three independently selected R₃₀ groups;
Q is OH, dialkylamino, 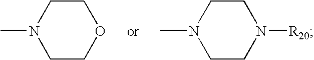
R₂₀ is selected from the group consisting of H, C₁₋₃ alkyl, and —CO(C₁₋₃ alkyl); and
R₃₀ is selected from the group consisting of dialkylamino, —CN and —OCF₃;
provided that:
i) when each R₅ is H, R₆ is H, n₅ is 0, and R₈ is H, then R₇ cannot be chlorine;
ii) when each R₅ is H, R₆ is H, n₅ is 0, X² is O or —CH₂—, and R₈ is H, then R₇ cannot be CH₃;
iii) when each R₅ is H, and R₆ is H, then R₇ and R₈ cannot both be fluorine;
iv) when each R₅ is H, R₆ is H, and X² is O, then R₇ and R₈ cannot both be chlorine;
v) when each R₅ is H, R₆ is H, X² is O, and R₈ is NO₂, then R₇ cannot be fluorine; and
vi) when each R₅ is H, R₆ is H, X² is SO₂, and R₈ is H, then R₇ cannot be fluorine or chlorine; and
b) a carrier or excipient system comprising:
i) about 10 to about 50% a first solubilizer by weight of the composition;
ii) about 10 to about 50% a second solubilizer by weight of the composition; and
iii) about 10 to about 30% a diluent by weight of the composition.

13. The pharmaceutical composition of claim 12, wherein the compound of Formula II has the Formula III: 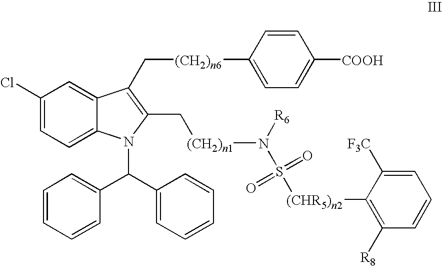
or a pharmaceutically acceptable salt thereof, wherein:
n₁ is 1 or 2;
n₂ is 1 or 2;
n₆ is 1 or 2;
R₅ is H or CH₃;
R₆ is H or C₁₋₆ alkyl; and
R₈ is selected from the group consisting of H, —OH, —NO₂, —CF₃, —OCF₃, —OCH₃, halogen, —COCH₃, —COOCH₃, dimethylamino, diethylamino and —CN.

14. The pharmaceutical composition of claim 12, wherein the compound of Formula II is 4-(3-{5-chloro-1-(diphenylmethyl)-2-[2-({[2(trifluoromethyl)benzyl]sulfonyl}amino)ethyl]-1H-indol-3-yl}propyl)benzoic acid or a pharmaceutically acceptable salt thereof.

15. The pharmaceutical composition of claim 12, wherein said pharmaceutically effective amount of said active pharmacological agent is about 0.1 to about 25% by weight of the composition.

16. The pharmaceutical composition of claim 12, wherein said first solubilizer is selected from the group consisting of polyethylene glycol 660 hydroxystearate, Vitamin E polyethylene glycol succinate, and mixtures thereof.

17. The pharmaceutical composition of claim 12, wherein said first solubilizer comprises polyethylene glycol 660 hydroxystearate.

18. The pharmaceutical composition of claim 12 wherein said second solubilizer is selected from the group consisting of polyoxyl 35 castor oil, polyoxyl 40 hydrogenated castor oil, polysorbate 80, and mixtures thereof.

19. The pharmaceutical composition of claim 12, wherein said second solubilizer comprises polyoxyl 35 castor oil.

20. The pharmaceutical composition of claim 12, wherein said diluent is selected from the group consisting of propylene glycol monocaprylate, a caprylocaproyl polyoxyglyceride, a medium chain monoglyceride, a medium chain diglyceride, a triglyceride of caprylic acid, a triglyceride of capric acid, a polyethylene glycol, propylene glycol, propylene carbonate, and mixtures thereof.

21. The pharmaceutical composition of claim 12, wherein said diluent comprises propylene glycol monocaprylate.

22. The pharmaceutical composition of claim 12 wherein said carrier or excipient system comprises:
i) a first solubilizer selected from the group consisting of polyethylene glycol 660 hydroxystearate, Vitamin E polyethylene glycol succinate, and mixtures thereof,
ii) a second solubilizer selected from the group consisting of polyoxyl 35 Castor oil, polyoxyl 40 hydrogenated castor oil, polysorbate 80, and mixtures thereof, and
iii) a diluent selected from the group consisting of propylene glycol monocaprylate, a caprylocaproyl polyoxyglyceride, a medium chain monoglyceride, a medium chain diglyceride a triglyceride of caprylic acid, a triglyceride of capric acid, a polyethylene glycol, propylene glycol, propylene carbonate, and mixtures thereof.

23. The pharmaceutical composition of claim 12 wherein said carrier or excipient system comprises:
i) about 10 to about 50% polyethylene glycol 660 hydroxystearate by weight of the composition;
ii) about 10 to about 50% polyoxyl 35 castor oil by weight of the composition; and
iii) about 10 to about 30% propylene glycol monocaprylate by weight of the composition.

24. A dosage form comprising a pharmaceutical composition of claim 12, wherein the composition contains from about 1 mg to about 125 mg of active pharmacological agent.

25. A dosage form comprising a pharmaceutical composition of claim 12, wherein the composition contains from about 3 mg to about 7 mg of active pharmacological agent.

26. A dosage form comprising a pharmaceutical composition of claim 12, wherein the composition contains from about 8 mg to about 12 mg of active pharmacological agent.

27. A dosage form comprising a pharmaceutical composition of claim 12, wherein the composition contains from about 13 mg to about 19 mg of active pharmacological agent.

28. A dosage form comprising a pharmaceutical composition of claim 12, wherein the composition contains from about 20 mg to about 30 mg of active pharmacological agent.

29. A dosage form comprising a pharmaceutical composition of claim 12, wherein the composition contains from about 31 mg to about 60 mg of active pharmacological agent.

30. A dosage form comprising a pharmaceutical composition of claim 12, wherein the composition contains from about 61 mg to about 80 mg of active pharmacological agent.

31. A dosage form comprising a pharmaceutical composition of claim 12, wherein the composition contains from about 81 mg to about 125 mg of active pharmacological agent.

32. A pharmaceutical composition comprising:
a) about 20% by weight of the composition of the an active pharmacological agent comprising 4-(3-{5-chloro-1-(diphenylmethyl)-2-[2-({[2(trifluoromethyl)benzyl]sulfonyl}amino)ethyl]-1H-indol-3-yl}propyl)benzoic acid or a pharmaceutically acceptable salt thereof, and
b) a carrier or excipient system comprising:
i) about 30% by weight of the composition of polyethylene glycol 660 hydroxystearate;
ii) about 30% by weight of the composition of polyoxyl 35 castor oil; and
iii) about 20% by weight of the composition of propylene glycol monocaprylate.

33. A dosage form comprising a pharmaceutical composition of claim 32, wherein said composition comprises about 100 mg of said active pharmacological agent.

34. A pharmaceutical composition comprising:
a) 2% by weight of the composition of an active pharmacological agent comprising 4-(3-{5-chloro-1-(diphenylmethyl)-2-[2-({[2(trifluoromethyl)benzyl]sulfonyl}amino)ethyl]-1H-indol-3-yl}propyl)benzoic acid or a pharmaceutically acceptable salt thereof; and
b) a carrier or excipient system comprising:
i) about 36% to about 37% by weight of the composition of polyethylene glycol 660 hydroxystearate;
ii) about 36% to about 37% by weight of the composition of polyoxyl 35 castor oil; and
iii) about 24% to about 25% by weight of the composition of propylene glycol monocaprylate.

35. The pharmaceutical composition of claim 34 comprising about 10 mg of the active pharmacological agent.

36. A process for preparing a pharmaceutical composition comprising:
a) a pharmaceutically effective amount of an active pharmacological agent having the Formula II: 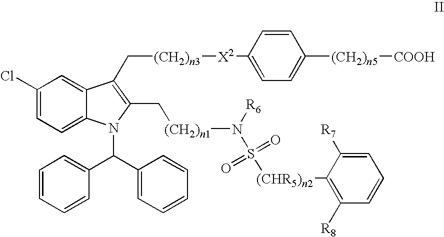
or a pharmaceutically acceptable salt thereof, wherein:
n₁ is 1 or 2;
n₂ is 1 or 2;
n₃ is 1 or 2;
n₅ is 0, 1 or 2;
X² is O, —CH₂— or SO₂;
each R₅ is independently H or C₁₋₃ alkyl;
R₆ is H or C₁₋₆ alkyl;
R₇ is selected from the group consisting of —OH, benzyloxy, —CH₃, —CF₃, —OCF₃, C₁₋₃ alkoxy, halogen, —CHO, —CO(C₁₋₃ alkyl), —CO(OC₁₋₃ alkyl), quinoline-5-yl, 3,5-dimethylisoxazol-4-yl, thiophene-3-yl, pyridin-4-yl, pyridine-3-yl, —CH₂-Q, and phenyl optionally substituted by from one to three independently selected R₃₀ groups;
R₈ is selected from the group consisting of H, —OH, —NO₂, —CF₃, —OCF₃, C₁₋₃ alkoxy, halogen, —CO(C₁₋₃ alkyl), —CO(OC₁₋₃ alkyl), quinoline-5-yl, 3,5-dimethylisoxazol-4-yl, thiophene-3-yl, —CH₂-Q, and phenyl substituted by from one to three independently selected R₃₀ groups;
Q is OH, dialkylamino, 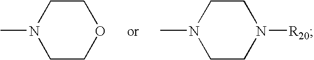
R₂₀ is selected from the group consisting of H, C₁₋₃ alkyl, and —CO(C₁₋₃ alkyl); and
R₃₀ is selected from the group consisting of dialkylamino, —CN, and —OCF₃;
provided that:
i) when each R₅ is H, R₆ is H, n₅ is 0, and R₈ is H, then R₇ cannot be chlorine;
ii) when each R₅ is H, R₆ is H, n₅ is 0, X² is O or —CH₂—, and R₈ is H, then R₇ cannot be CH₃;
iii) when each R₅ is H, and R₆ is H, then R₇ and R₈ cannot both be fluorine;
iv) when each R₅ is H, R₆ is H, and X² is O, then R₇ and R₈ cannot both be chlorine;
v) when each R₅ is H, R₆ is H, X² is O, and R₈ is NO₂, then R₇ cannot be fluorine; and
vi) when each R₅ is H, R₆ is H, X² is SO₂, and R₈ is H, then R₇ cannot be fluorine or chlorine; and
b) a carrier or excipient system comprising:
i) about 10 to about 50% a first solubilizer by weight of the composition;
ii) about 10 to about 50% a second solubilizer by weight of the composition; and
iii) about 10 to about 30% a diluent by weight of the composition;
said process comprising:
(1) mixing the first solubilizer, second solubilizer, and diluent to form a first homogenous solution;
(2) adding the pharmacological agent or a pharmaceutically acceptable salt thereof to the first homogenous solution; and
(3) mixing the pharmacological agent and the first homogenous solution at a temperature sufficient to facilitate dissolution of the pharmacological agent to obtain a second homogenous solution.

37. The process of claim 36, wherein step (1) further comprises heating the first solubilizer, second solubilizer, and diluent to a temperature sufficient to form the first homogenous solution.

38. The process of claim 37, wherein said mixing of the first solubilizer, second solubilizer, and diluent is performed at a temperature of from about 80° C. to about 90° C.

39. The process of claim 36, wherein the mixing of the pharmacologically active agent in step (3) is performed at a temperature of from about 80° C. to about 90° C.

40. The process of claim 36 further comprising encapsulating at least a portion of said second homogenous solution into one or more unit dosage capsule forms.

41. The process of claim 40, wherein prior to encapsulation, said second homogenous solution is screened to remove undissolved particles.

42. The process of claim 40, wherein prior to encapsulation, said third homogenous solution is cooled.

43. The process of claim 36, wherein the pharmaceutically effective amount of the active pharmacological agent is about 0.1 to about 20% by weight of the composition.

44. The process of claim 36, wherein the first solubilizer is selected from the group consisting of polyethylene glycol 660 hydroxystearate, Vitamin E polyethylene glycol succinate, and mixtures thereof.

45. The process of claim 36, wherein the first solubilizer comprises polyethylene glycol 660 hydroxystearate.

46. The process of claim 36, wherein the second solubilizer is selected from the group consisting of polyoxyl 35 castor oil, polyoxyl 40 hydrogenated castor oil, polysorbate 80, and mixtures thereof.

47. The process of claim 36, wherein the second solubilizer comprises polyoxyl 35 castor oil.

48. The process of claim 36, wherein the diluent is selected from the group consisting of propylene glycol monocaprylate, caprylocaproyl polyoxyglycerides, a medium chain monoglyceride, a medium chain diglyceride, a triglyceride of caprylic acid, a triglyceride of capric acid, a polyethylene glycol, propylene glycol, propylene carbonate, and mixtures thereof.

49. The process of claim 36 wherein the diluent comprises propylene glycol monocaprylate.

50. The process of claim 36, wherein said carrier or excipient system comprises:
i) a first solubilizer selected from the group consisting of polyethylene glycol 660 hydroxystearate, vitamin E polyethylene glycol succinate, and mixtures thereof,
ii) a second solubilizer selected from the group consisting of polyoxyl 35 castor oil, polyoxyl 40 hydrogenated castor oil, polysorbate 80, and mixtures thereof, and
iii) a diluent selected from the group consisting of propylene glycol monocaprylate, a caprylocaproyl polyoxyglyceride, a medium chain monoglyceride, a medium chain diglyceride a triglyceride of caprylic acid, a triglyceride of capric acid, a polyethylene glycol, propylene glycol, propylene carbonate, and mixtures thereof.

51. The process of claim 36, wherein said carrier or excipient system comprises:
i) about 10 to about 50% polyethylene glycol 660 hydroxystearate by weight of the composition;
ii) about 10 to about 50% polyoxyl 35 castor oil by weight of the composition; and
iii) about 10 to about 30% propylene glycol monocaprylate by weight of the composition.

52. The process of claim 36, wherein the active pharmacological agent of Formula II has the Formula III: 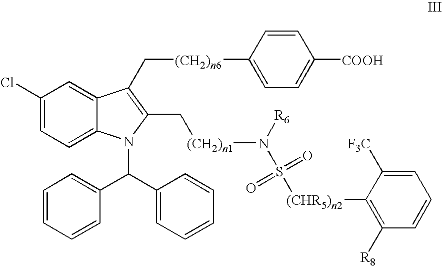
or a pharmaceutically acceptable salt thereof, wherein:
n₁ is 1 or 2;
n₂ is 1 or 2;
n₆ is 1 or 2;
R₅ is H or CH₃;
R₆ is H or C₁₋₆ alkyl; and
R₈ is selected from the group consisting of H, —OH, —NO₂, —CF₃, —OCF₃, —OCH₃, halogen, —COCH₃, —COOCH₃, dimethylamino, diethylamino, and —CN.

53. The process of claim 36, wherein the active pharmacological agent comprises 4-(3-{5-chloro-1-(diphenylmethyl)-2-[2-({[2-(trifluoromethyl)benzyl]sulfonyl}amino)ethyl]-1H-indol-3-yl}propyl)benzoic acid or a pharmaceutically acceptable salt thereof.

54. A process for preparing a pharmaceutical composition comprising:
a) 20% by weight of the composition of an active pharmacological agent comprising 4-(3-{5-chloro-1-(diphenylmethyl)-2-[2-({[2(trifluoromethyl)benzyl]sulfonyl}amino)ethyl]-1H-indol-3-yl}propyl)benzoic acid or a pharmaceutically acceptable salt thereof, and
b) a carrier or excipient system comprising:
i) about 30% polyethylene glycol 660 hydroxystearate by weight of the composition;
ii) about 30% polyoxyl 35 castor oil by weight of the composition; and
iii) about 20% propylene glycol monocaprylate by weight of the composition;
said process comprising:
(1) mixing the polyethylene glycol 660 hydroxystearate, polyoxyl 35 castor oil, and propylene glycol monocaprylate to form a first homogenous solution;
(2) adding the pharmacological agent or a pharmaceutically acceptable salt thereof to the first homogenous solution;
(3) mixing the pharmacological agent and the first homogenous solution at a temperature sufficient to facilitate dissolution of said pharmacological agent to obtain a second homogenous solution.

55. The process of claim 54, wherein step (1) further comprises heating the polyethylene glycol 660 hydroxystearate, polyoxyl 35 castor oil, and propylene glycol monocaprylate to a temperature sufficient to form the first homogenous solution.

56. The process of claim 55, wherein said mixing of the polyethylene glycol 660 hydroxystearate, polyoxyl 35 castor oil, and propylene glycol monocaprylate is performed at a temperature of from about 80° C. to about 90° C.

57. The process of claim 54, wherein the mixing of the pharmacologically active agent in step (3) is performed at a temperature of from about 80° C. to about 90° C.

58. The process of claim 54, further comprising encapsulating at least a portion of said second homogenous solution into one or more unit dosage capsule forms.

59. The process of claim 58, wherein prior to encapsulation, the second homogenous solution is screened to remove undissolved particles.

60. The process of claim 58, wherein prior to encapsulation, the second homogenous solution is cooled.

61. A process for preparing a pharmaceutical composition comprising:
a) 2% by weight of the composition of an active pharmacological agent comprising 4-(3-{5-chloro-1-(diphenylmethyl)-2-[2-({[2(trifluoromethyl)benzyl]sulfonyl}amino)ethyl]-1H-indol-3-yl}propyl)benzoic acid or a pharmaceutically acceptable salt thereof; and
b) a carrier or excipient system comprising:
i) about 36% to about 37% by weight of the composition of polyethylene glycol 660 hydroxystearate;
ii) about 36% to about 37% by weight of the composition of polyoxyl 35 castor oil; and
iii) about 24% to about 25% by weight of the composition of propylene glycol monocaprylate.
said process comprising:
(1) mixing the polyethylene glycol 660 hydroxystearate, polyoxyl 35 castor oil, and propylene glycol monocaprylate to form a first homogenous solution;
(2) adding the pharmacological agent or a pharmaceutically acceptable salt thereof to the first homogenous solution;
(3) mixing the pharmacological agent and the first homogenous solution at a temperature sufficient to facilitate dissolution of the pharmacological agent to obtain a second homogenous solution.

62. The process of claim 61, wherein step (1) further comprises heating the polyethylene glycol 660 hydroxystearate, polyoxyl 35 castor oil, and propylene glycol monocaprylate to a temperature sufficient to form the first homogenous solution.

63. The process of claim 62, wherein said mixing of the polyethylene glycol 660 hydroxystearate, polyoxyl 35 castor oil, and propylene glycol monocaprylate is performed at a temperature of from about 80° C. to about 90° C.

64. The process of claim 61, wherein the mixing of the pharmacologically active agent in step (3) is performed at a temperature of from about 80° C. to about 90° C.

65. The process of claim 61, further comprising encapsulating at least a portion of said second homogenous solution into one or more unit dosage capsule forms.

66. The process of claim 65, wherein prior to encapsulation, said second homogenous solution is screened to remove undissolved particles.

67. The process of claim 65, wherein prior to encapsulation, the second homogenous solution is cooled.

68. A product made by the process of claim 36.
